SEQUENCE LISTING (86 sequences)

SEQ ID NO: 1 - protein, 696 aa, Homo sapiens
MKFAEHLSAHITPEWRKQYIQYEAFKDMLYSAQDQAPSVEVTDEDTVKRYFAKFEEKFFQTCEKELAKINTFYSEKLAEAQRRFATLQNELQSSLDAQKESTGVTTLRQRRKPVFHLSHEERVQHRNIKDLKLAFSEFYLSLILLQNYQNLNFTGFRKILKKHDKILETSRGADWRVAHVEVAPFYTCKKINQLISETEAVVTNELEDGDRQKAMKRLRVPPLGAAQPAPAWTTFRVGLFCGIFIVLNITLVLAAVFKLETDRSIWPLIRIYRGGFLLIEFLFLLGINTYGWRQAGVNHVLIFELNPRSNLSHQHLFEIAGFLGILWCLSLLACFFAPISVIPTYVYPLALYGFMVFFLINPTKTFYYKSRFWLLKLLFRVFTAPFHKVGFADFWLADQLNSLSVILMDLEYMICFYSLELKWDESKGLLPNNSEESGICHKYTYGVRAIVQCIPAWLRFIQCLRRYRDTKRAFPHLVNAGKYSTTFFMVTFAALYSTHKERGHSDTMVFFYLWIVFYIISSCYTLIWDLKMDWGLFDKNAGENTFLREEIVYPQKAYYYCAIIEDVILRFAWTIQISITSTTLLPHSGDIIATVFAPLEVFRRFVWNFFRLENEHLNNCGEFRAVRDISVAPLNADDQTLLEQMMDQDDGVRNRQKNRSWKYNQSISLRRPRLASQSKARDTKVLIEDTDDEANT

SEQ ID NO: 2 - protein, 564 aa, Homo sapiens
MGNWVVNHWFSVLFLVVWLGLNVFLFVDAFLKYEKADKYYYTRKILGSTLACARASALCLNFNSTLILLPVCRNLLSFLRGTCSFCSRTLRKQLDHNLTFHKLVAYMICLHTAIHIIAHLFNFDCYSRSRQATDGSLASILSSLSHDEKKGGSWLNPIQSRNTTVEYVTFTSIAGLTGVIMTIALILMVTSATEFIRRSYFEVFWYTHHLFIFYILGLGIHGIGGIVRGQTEESMNESHPRKCAESFEMWDDRDSHCRRPKFEGHPPESWKWILAPVILYICERILRFYRSQQKVVITKVVMHPSKVLELQMNKRGFSMEVGQYIFVNCPSISLLEWHPFTLTSAPEEDFFSIHIRAAGDWTENLIRAFEQQYSPIPRIEVDGPFGTASEDVFQYEVAVLVGAGIGVTPFASILKSIWYKFQCADHNLKTKKIYFYWICRETGAFSWFNNLLTSLEQEMEELGKVGFLNYRLFLTGWDSNIVGHAALNFDKATDIVTGLKQKTSFGRPMWDNEFSTIATSHPKSVVGVFLCGPRTLAKSLRKCCHRYSSLDPRKVQFYFNKENF

SEQ ID NO: 3 - protein, 410 aa, Homo sapiens
MEDPSGAREPRARPRERDPGRRPHPDQGRTHDRPRDRPGDPRRKRSSDGNRRRDGDRDPERDQERDGNRDRNRDRERERERERDPDRGPRRDTHRDAGPRAGEHGVWEKPRQSRTRDGARGLTWDAAAPPGPAPWEAPEPPQPQRKGDPGRRRPESEPPSERYLPSTPRPGREEVEYYQSEAEGLLECHKCKYLCTGRGVVQIVEVVLNGMVLICIVASYFVLAGFSASFSSGGGFGNNYYSPFEGTELEQVRQLDQQYTILRSPLIYGGVAVSLGLGVLTMGVLLQGAKSRTMLSGKWLLTEAAFSLLAAVGYCTGIGVYLHVALQINSTDTCKTRERLYARKGLTWMDCQLAGTDGAAATFACLLVIMYGASVVLALRSYREQKRYKGSREQPGSYSDAPEYLWSGTL

SEQ ID NO: 4 - protein, 401 aa, Homo sapiens
MEDPSGAREPRARPRERDPGRRPHPDQGRTHDRPRDRPGDPRRKRSSDGNRRRDGDRDPERDQERDGNRDRNRDRERERERERDPDRGPRRDTHRDAGPRAGEHGVWEKPRQSRTRDGARGLTWDAAAPPGPAPWEAPEPPQPQRKGDPGRRRPESEPPSERYLPSTPRPGREEVEYYQSEAEGLLECHKCKYLCTGRACCQMLEVLLNLLILACSSVSYSSTGGYTGITSLGGIYYYQFGGAYSGFDGADGEKAQQLDVQFYQLKLPMVTVAMACSGALTALCCLFVAMGVLRVPWHCPLLLVTEGLLDMLIAGGYIPALYFYFHYLSAAYGSPVCKERQALYQSKGYSGFGCSFHGADIGAGIFAALGIVVFALGAVLAIKGYRKVRKLKEKPAEMFEF

SEQ ID NO: 5 - protein, 252 aa, Homo sapiens
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 6 - protein, 330 aa, Homo sapiens [sequence omitted]

SEQ ID NO: 7 - protein, 431 aa, Homo sapiens
MFFGGEGSLTYTLVIICFLTLRLSASQNCLKKSLEDVVIDIQSSLSKGIRGNEPVYTSTQEDCINSCCSTKNISGDKACNLMIFDTRKTARQPNCYLFFCPNEEACPLKPAKGLMSYRIITDFPSLTRNLPSQELPQEDSLLHGQFSQAVTPLAHHHTDYSKPTDISWRDTLSQKFGSSDHLEKLFKMDEASAQLLAYKEKGHSQSSQFSSDQEIAHLLPENVSALPATVAVASPHTTSATPKPATLLPTNASVTPSGTSQPQLATTAPPVTTVTSQPPTTLISTVFTRAAATLQAMATTAVLTTTFQAPTDSKGSLETIPFTEISNLTLNTGNVYNPTALSMSNVESSTMNKTASWEGREASPGSSSQGSVPENQYGLPFEKWLLIGSLLFGVLFLVIGLVLLGRILSESLRRKRYSRLDYLINGIYVDI

SEQ ID NO: 8 - protein, 1212 aa, Homo sapiens
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 9 - protein, 836 aa, Homo sapiens
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 10 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 11 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 12 - protein, 910 aa, Homo sapiens
MGTPRAQHPPPPQLLFLILLSCPWIQGLPLKEEEILPEPGSETPTVASEALAELLHGALLRRGPEMGYLPGSDRDPTLATPPAGQTLAVPSLPRATEPGTGPLTTAVTPNGVRGAGPTAPELLTPPPGTTAPPPPSPASPGPPLGPEGGEEETTTTIITTTTVTTTVTSPVLCNNNISEGEGYVESPDLGSPVSRTLGLLDCTYSIHVYPGYGIEIQVQTLNLSQEEELLVLAGGGSPGLAPRLLANSSMLGEGQVLRSPTNRLLLHFQSPRVPRGGGFRIHYQAYLLSCGFPPRPAHGDVSVTDLHPGGTATFHCDSGYQLQGEETLICLNGTRPSWNGETPSCMASCGGTIHNATLGRIVSPEPGGAVGPNLTCRWVIEAAEGRRLHLHFERVSLDEDNDRLMVRSGGSPLSPVIYDSDMDDVPERGLISDAQSLYVELLSETPANPLLLSLRFEAFEEDRCFAPFLAHGNVTTTDPEYRPGALATFSCLPGYALEPPGPPNAIECVDPTEPHWNDTEPACKAMCGGELSEPAGVVLSPDWPQSYSPGQDCVWGVHVQEEKRILLQVEILNVREGDMLTLFDGDGPSARVLAQLRGPQPRRRLLSSGPDLTLQFQAPPGPPNPGLGQGFVLHFKEVPRNDTCPELPPPEWGWRTASHGDLIRGTVLTYQCEPGYELLGSDILTCQWDLSWSAAPPACQKIMTCADPGEIANGHRTASDAGFPVGSHVQYRCLPGYSLEGAAMLTCYSRDTGTPKWSDRVPKCALKYEPCLNPGVPENGYQTLYKHHYQAGESLRFFCYEGFELIGEVTITCVPGHPSQWTSQPPLCKVTQTTDPSRQLEGGNLALAILLPLGLVIVLGSGVYIYYTKLQGKSLFGFSGSHSYSPITVESDFSNPLYEAGDTREYEVSI

SEQ ID NO: 13 - protein, 555 aa, Homo sapiens
MARPDDEEGAAVAPGHPLAKGYLPLPRGAPVGKESVELQNGPKAGTFPVNGAPRDSLAAASGVLGGPQTPLAPEEETQARLLPAGAGAETPGAESSPLPLTALSPRRFVVLLIFSLYSLVNAFQWIQYSIISNVFEGFYGVTLLHIDWLSMVYMLAYVPLIFPATWLLDTRGLRLTALLGSGLNCLGAWIKCGSVQQHLFWVTMLGQCLCSVAQVFILGLPSRIASVWFGPKEVSTACATAVLGNQLGTAVGFLLPPVLVPNTQNDTNLLACNISTMFYGTSAVATLLFILTAIAFKEKPRYPPSQAQAALQDSPPEEYSYKKSIRNLFKNIPFVLLLITYGIMTGAFYSVSTLLNQMILTYYEGEEVNAGRIGLTLVVAGMVGSILCGLWLDYTKTYKQTTLIVYILSFIGMVIFTFTLDLRYIIIVFVTGGVLGFFMTGYLPLGFEFAVEITYPESEGTSSGLLNASAQIFGILFTLAQGKLTSDYGPKAGNIFLCVWMFIGIILTALIKSDLRRHNINIGITNVDVKAIPADSPTDQEPKTVMLSKQSESAI

SEQ ID NO: 14 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 15 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 16 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 17 - protein, 655 aa, Homo sapiens
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 18 - protein, 437 aa, Homo sapiens
MLQDPDSDQPLNSLDVKPLRKPRIPMETFRKVGIPIIIALLSLASIIIVVVLIKVILDKYYFLCGQPLHFIPRKQLCDGELDCPLGEDEEHCVKSFPEGPAVAVRLSKDRSTLQVLDSATGNWFSACFDNFTEALAETACRQMGYSSKPTFRAVEIGPDQDLDVVEITENSQELRMRNSSGPCLSGSLVSLHCLACGKSLKTPRVVGGEEASVDSWPWQVSIQYDKQHVCGGSILDPHWVLTAAHCFRKHTDVFNWKVRAGSDKLGSFPSLAVAKIIIIEFNPMYPKDNDIALMKLQFPLTFSGTVRPICLPFFDEELTPATPLWIIGWGFTKQNGGKMSDILLQASVQVIDSTRCNADDAYQGEVTEKMMCAGIPEGGVDTCQGDSGGPLMYQSDQWHVVGIVSWGYGCGGPSTPGVYTKVSAYLNWIYNVWKAEL

SEQ ID NO: 19 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 21 aa, Artificial sequence
  an artificially synthesized sequence
FNNFTVSFWLRVPKVSASHLE

SEQ ID NO: 23 - DNA, 2154 bp, Artificial sequence
  an artificially synthesized sequence
atgaagttcgccgagcacctctccgcgcacatcactcccgagtggaggaagcaatacatccagtatgaggctttcaaggatatgctgtattcagctcaggaccaggcaccttctgtggaagttacagatgaggacacagtaaagaggtattttgccaagtttgaagagaagtttttccaaacctgtgaaaaagaacttgccaaaatcaacacattttattcagagaagctcgcagaggctcagcgcaggtttgctacacttcagaatgagcttcagtcatcactggatgcacagaaagaaagcactggtgttactacgctgcgacaacgcagaaagccagtcttccacttgtcccatgaggaacgtgtccaacatagaaatattaaagaccttaaactggccttcagtgagttctacctcagtctaatcctgctgcagaactatcagaatctgaattttacagggtttcgaaaaatcctgaaaaagcatgacaagatcctggaaacatctcgtggagcagattggcgagtggctcacgtagaggtggccccattttatacatgcaagaaaatcaaccagcttatctctgaaactgaggctgtagtgaccaatgaacttgaagatggtgacagacaaaaggctatgaagcgtttacgtgtcccccctttgggagctgctcagcctgcaccagcatggactacttttagagttggcctattttgtggaatattcattgtactgaatattacccttgtgcttgccgctgtatttaaacttgaaacagatagaagtatatggcccttgataagaatctatcggggtggctttcttctgattgagttcctttttctactgggcatcaacacgtatggttggagacaggctggagtaaaccatgtactcatctttgaacttaatccgagaagcaatttgtctcatcaacatctctttgagattgctggattcctcgggatattgtggtgcctgagccttctggcatgcttctttgctccaattagtgtcatccccacatatgtgtatccacttgccctttatggatttatggttttcttccttatcaaccccaccaaaactttctactataaatcccggttttggctgcttaaactgctgtttcgagtatttacagcccccttccataaggtaggctttgctgatttctggctggcggatcagctgaacagcctgtcagtgatactgatggacctggaatatatgatctgcttctacagtttggagctcaaatgggatgaaagtaagggcctgttgccaaataattcagaagaatcaggaatttgccacaaatatacatatggtgtgcgggccattgttcagtgcattcctgcttggcttcgcttcatccagtgcctgcgccgatatcgagacacaaaaagggcctttcctcatttagttaatgctggcaaatactccacaactttcttcatggtgacgtttgcagccctttacagcactcacaaagaacgaggtcactcggacactatggtgttcttttacctgtggattgtcttttatatcatcagttcctgctataccctcatctgggatctcaagatggactggggtctcttcgataagaatgcaggagagaacactttcctccgggaagagattgtatacccccaaaaagcctactactactgtgccataatagaggatgtgattctgcgctttgcttggactatccaaatctcgattacctctacaactttgttgcctcattctggggacatcattgctactgtctttgccccacttgaggttttccggcgatttgtgtggaacttcttccgcctggagaatgaacatctgaataactgtggtgagttccgtgctgtgcgggacatctctgtggcccccctgaacgcagatgatcagactctcctagaacagatgatggaccaggatgatggggtacgaaaccgccagaagaatcggtcatggaagtacaaccagagcatatccctgcgccggcctcgcctcgcttctcaatccaaggctcgtgacactaaggtattgatagaagacacagatgatgaagctaacactttcaacaacttcacagtgtctttctggctgcgagtgcccaaggtgtctgcctcccatctggaatga

SEQ ID NO: 24 - protein, 717 aa, Artificial sequence
  an artificially synthesized sequence
MKFAEHLSAHITPEWRKQYIQYEAFKDMLYSAQDQAPSVEVTDEDTVKRYFAKFEEKFFQTCEKELAKINTFYSEKLAEAQRRFATLQNELQSSLDAQKESTGVTTLRQRRKPVFHLSHEERVQHRNIKDLKLAFSEFYLSLILLQNYQNLNFTGFRKILKKHDKILETSRGADWRVAHVEVAPFYTCKKINQLISETEAVVTNELEDGDRQKAMKRLRVPPLGAAQPAPAWTTFRVGLFCGIFIVLNITLVLAAVFKLETDRSIWPLIRIYRGGFLLIEFLFLLGINTYGWRQAGVNHVLIFELNPRSNLSHQHLFEIAGFLGILWCLSLLACFFAPISVIPTYVYPLALYGFMVFFLINPTKTFYYKSRFWLLKLLFRVFTAPFHKVGFADFWLADQLNSLSVILMDLEYMICFYSLELKWDESKGLLPNNSEESGICHKYTYGVRAIVQCIPAWLRFIQCLRRYRDTKRAFPHLVNAGKYSTTFFMVTFAALYSTHKERGHSDTMVFFYLWIVFYIISSCYTLIWDLKMDWGLFDKNAGENTFLREEIVYPQKAYYYCAIIEDVILRFAWTIQISITSTTLLPHSGDIIATVFAPLEVFRRFVWNFFRLENEHLNNCGEFRAVRDISVAPLNADDQTLLEQMMDQDDGVRNRQKNRSWKYNQSISLRRPRLASQSKARDTKVLIEDTDDEANTFNNFTVSFWLRVPKVSASHLE

SEQ ID NO: 25 - protein, 10 aa, Artificial sequence
  an artificially synthesized sequence
EQKLISEEDL

SEQ ID NO: 26 - DNA, 2121 bp, Artificial sequence
  an artificially synthesized sequence
atgaagttcgccgagcacctctccgcgcacatcactcccgagtggaggaagcaatacatccagtatgaggctttcaaggatatgctgtattcagctcaggaccaggcaccttctgtggaagttacagatgaggacacagtaaagaggtattttgccaagtttgaagagaagtttttccaaacctgtgaaaaagaacttgccaaaatcaacacattttattcagagaagctcgcagaggctcagcgcaggtttgctacacttcagaatgagcttcagtcatcactggatgcacagaaagaaagcactggtgttactacgctgcgacaacgcagaaagccagtcttccacttgtcccatgaggaacgtgtccaacatagaaatattaaagaccttaaactggccttcagtgagttctacctcagtctaatcctgctgcagaactatcagaatctgaattttacagggtttcgaaaaatcctgaaaaagcatgacaagatcctggaaacatctcgtggagcagattggcgagtggctcacgtagaggtggccccattttatacatgcaagaaaatcaaccagcttatctctgaaactgaggctgtagtgaccaatgaacttgaagatggtgacagacaaaaggctatgaagcgtttacgtgtcccccctttgggagctgctcagcctgcaccagcatggactacttttagagttggcctattttgtggaatattcattgtactgaatattacccttgtgcttgccgctgtatttaaacttgaaacagatagaagtatatggcccttgataagaatctatcggggtggctttcttctgattgagttcctttttctactgggcatcaacacgtatggttggagacaggctggagtaaaccatgtactcatctttgaacttaatccgagaagcaatttgtctcatcaacatctctttgagattgctggattcctcgggatattgtggtgcctgagccttctggcatgcttctttgctccaattagtgtcatccccacatatgtgtatccacttgccctttatggatttatggttttcttccttatcaaccccaccaaaactttctactataaatcccggttttggctgcttaaactgctgtttcgagtatttacagcccccttccataaggtaggctttgctgatttctggctggcggatcagctgaacagcctgtcagtgatactgatggacctggaatatatgatctgcttctacagtttggagctcaaatgggatgaaagtaagggcctgttgccaaataattcagaagaatcaggaatttgccacaaatatacatatggtgtgcgggccattgttcagtgcattcctgcttggcttcgcttcatccagtgcctgcgccgatatcgagacacaaaaagggcctttcctcatttagttaatgctggcaaatactccacaactttcttcatggtgacgtttgcagccctttacagcactcacaaagaacgaggtcactcggacactatggtgttcttttacctgtggattgtcttttatatcatcagttcctgctataccctcatctgggatctcaagatggactggggtctcttcgataagaatgcaggagagaacactttcctccgggaagagattgtatacccccaaaaagcctactactactgtgccataatagaggatgtgattctgcgctttgcttggactatccaaatctcgattacctctacaactttgttgcctcattctggggacatcattgctactgtctttgccccacttgaggttttccggcgatttgtgtggaacttcttccgcctggagaatgaacatctgaataactgtggtgagttccgtgctgtgcgggacatctctgtggcccccctgaacgcagatgatcagactctcctagaacagatgatggaccaggatgatggggtacgaaaccgccagaagaatcggtcatggaagtacaaccagagcatatccctgcgccggcctcgcctcgcttctcaatccaaggctcgtgacactaaggtattgatagaagacacagatgatgaagctaacactgagcagaagctcatctcagaagaagacctctga

SEQ ID NO: 27 - protein, 706 aa, Artificial sequence
  an artificially synthesized sequence
MKFAEHLSAHITPEWRKQYIQYEAFKDMLYSAQDQAPSVEVTDEDTVKRYFAKFEEKFFQTCEKELAKINTFYSEKLAEAQRRFATLQNELQSSLDAQKESTGVTTLRQRRKPVFHLSHEERVQHRNIKDLKLAFSEFYLSLILLQNYQNLNFTGFRKILKKHDKILETSRGADWRVAHVEVAPFYTCKKINQLISETEAVVTNELEDGDRQKAMKRLRVPPLGAAQPAPAWTTFRVGLFCGIFIVLNITLVLAAVFKLETDRSIWPLIRIYRGGFLLIEFLFLLGINTYGWRQAGVNHVLIFELNPRSNLSHQHLFEIAGFLGILWCLSLLACFFAPISVIPTYVYPLALYGFMVFFLINPTKTFYYKSRFWLLKLLFRVFTAPFHKVGFADFWLADQLNSLSVILMDLEYMICFYSLELKWDESKGLLPNNSEESGICHKYTYGVRAIVQCIPAWLRFIQCLRRYRDTKRAFPHLVNAGKYSTTFFMVTFAALYSTHKERGHSDTMVFFYLWIVFYIISSCYTLIWDLKMDWGLFDKNAGENTFLREEIVYPQKAYYYCAIIEDVILRFAWTIQISITSTTLLPHSGDIIATVFAPLEVFRRFVWNFFRLENEHLNNCGEFRAVRDISVAPLNADDQTLLEQMMDQDDGVRNRQKNRSWKYNQSISLRRPRLASQSKARDTKVLIEDTDDEANTEQKLISEEDL

SEQ ID NO: 28 - DNA, 1206 bp, Homo sapiens
atggaagatccgtcgggggctcgcgagccccgggcccggccgagagagcgggacccgggacggcgcccccacccagaccaaggccgcacccacgatagaccgcgggaccgacccggggacccgcgcaggaagcgaagcagcgacgggaaccggcgaagggacggggaccgggacccggagagagaccaggagagggacgggaaccgcgaccggaaccgggaccgggagagggagagagagagggaaagagacccggaccgaggcccccgccgggacacacacagggacgcgggccctcgcgcaggtgaacacggagtttgggaaaaaccgcgccaaagccggacgcgggacggagcccggggactgacctgggacgcagccgcgcctcctgggcccgcgccctgggaagccccggagccgccgcagccgcagaggaagggagaccccgggcgccgcagacccgaaagtgaacccccttcggagagatatctgccctcgacccccaggcctggacgagaggaggtggaatattaccagtcagaggcggaaggactcctggaatgccacaaatgcaaatacttgtgcactgggagagcctgctgccaaatgctggaggttctcctgaacttgctgatcctggcctgcagctctgtgtcttacagttccacagggggctacacgggcatcaccagcttggggggcatttactactatcagttcggaggggcttacagtggctttgatggtgctgacggggagaaggcccagcaactggatgtccagttctaccagctaaagctgcccatggtcactgtggcaatggcctgtagtggagccctcacagccctctgctgcctcttcgttgccatgggtgtcctgcgggtcccgtggcattgtccactgttgctggtgaccgaaggcttgttggacatgctcatcgcgggggggtacatcccggccttgtacttctacttccactacctctctgctgcctatggctctcctgtgtgtaaagagaggcaggcgctgtaccaaagcaaaggctacagcggtttcggctgcagtttccacggagcagatataggagctggaatctttgctgccctgggcattgtggtctttgccctgggggctgtcctggccataaagggctaccgaaaagttaggaagctaaaagagaagccagcagaaatgtttgaattttga

SEQ ID NO: 29 - DNA, 1236 bp, Artificial sequence
  an artificially synthesized sequence
atggagcagaagctcatctcagaagaagacctcgaagatccgtcgggggctcgcgagccccgggcccggccgagagagcgggacccgggacggcgcccccacccagaccaaggccgcacccacgatagaccgcgggaccgacccggggacccgcgcaggaagcgaagcagcgacgggaaccggcgaagggacggggaccgggacccggagagagaccaggagagggacgggaaccgcgaccggaaccgggaccgggagagggagagagagagggaaagagacccggaccgaggcccccgccgggacacacacagggacgcgggccctcgcgcaggtgaacacggagtttgggaaaaaccgcgccaaagccggacgcgggacggagcccggggactgacctgggacgcagccgcgcctcctgggcccgcgccctgggaagccccggagccgccgcagccgcagaggaagggagaccccgggcgccgcagacccgaaagtgaacccccttcggagagatatctgccctcgacccccaggcctggacgagaggaggtggaatattaccagtcagaggcggaaggactcctggaatgccacaaatgcaaatacttgtgcactgggagagcctgctgccaaatgctggaggttctcctgaacttgctgatcctggcctgcagctctgtgtcttacagttccacagggggctacacgggcatcaccagcttggggggcatttactactatcagttcggaggggcttacagtggctttgatggtgctgacggggagaaggcccagcaactggatgtccagttctaccagctaaagctgcccatggtcactgtggcaatggcctgtagtggagccctcacagccctctgctgcctcttcgttgccatgggtgtcctgcgggtcccgtggcattgtccactgttgctggtgaccgaaggcttgttggacatgctcatcgcgggggggtacatcccggccttgtacttctacttccactacctctctgctgcctatggctctcctgtgtgtaaagagaggcaggcgctgtaccaaagcaaaggctacagcggtttcggctgcagtttccacggagcagatataggagctggaatctttgctgccctgggcattgtggtctttgccctgggggctgtcctggccataaagggctaccgaaaagttaggaagctaaaagagaagccagcagaaatgtttgaattttga

SEQ ID NO: 30 - protein, 411 aa, Artificial sequence
  an artificially synthesized sequence
MEQKLISEEDLEDPSGAREPRARPRERDPGRRPHPDQGRTHDRPRDRPGDPRRKRSSDGNRRRDGDRDPERDQERDGNRDRNRDRERERERERDPDRGPRRDTHRDAGPRAGEHGVWEKPRQSRTRDGARGLTWDAAAPPGPAPWEAPEPPQPQRKGDPGRRRPESEPPSERYLPSTPRPGREEVEYYQSEAEGLLECHKCKYLCTGRACCQMLEVLLNLLILACSSVSYSSTGGYTGITSLGGIYYYQFGGAYSGFDGADGEKAQQLDVQFYQLKLPMVTVAMACSGALTALCCLFVAMGVLRVPWHCPLLLVTEGLLDMLIAGGYIPALYFYFHYLSAAYGSPVCKERQALYQSKGYSGFGCSFHGADIGAGIFAALGIVVFALGAVLAIKGYRKVRKLKEKPAEMFEF

SEQ ID NO: 31 - protein, 28 aa, Artificial sequence
  an artificially synthesized sequence
WSHPQFEKGGGSGGGSGGSAWSHPQFEK

SEQ ID NO: 32 - DNA, 2175 bp, Artificial sequence
  an artificially synthesized sequence
atgtaccagaggatgctccggtgcggtgccgagctgggatcgcccggaggcggcagtagtggcggcgcaggggggcgcctggccctgctctggatagtcccgctcaccctcagcggcctcctaggagtggcctggggggcatccagtttgggagcgcaccacatccaccataaacttgactacaaagacgatgacgacaagggtgggttagccaaccccacccgagtgggcggccgcgaaccatacccaggctcggcagaggtgatccgggagtctagcagtaccactggcatggtggtggggattgtcgcagcagcagctctgtgcatcctcatcctcctctatgccatgaagaagcgcagggccaagggaggttcaggaggttcaggaggtctcgagcacggaactatcccattcaacagaactcacaggtccaagagaagctccggaatgggaaactgggtggttaaccactggttttcagttttgtttctggttgtttggttagggctgaatgttttcctgtttgtggatgccttcctgaaatatgagaaggccgacaaatactactacacaagaaaaatccttgggtcaacattggcctgtgcccgagcgtctgctctctgcttgaattttaacagcacgctgatcctgcttcctgtgtgtcgcaatctgctgtccttcctgaggggcacctgctcattttgcagccgcacactgagaaagcaattggatcacaacctcaccttccacaagctggtggcctatatgatctgcctacatacagctattcacatcattgcacacctgtttaactttgactgctatagcagaagccgacaggccacagatggctcccttgcctccattctctccagcctatctcatgatgagaaaaaggggggttcttggctaaatcccatccagtcccgaaacacgacagtggagtatgtgacattcaccagcattgctggtctcactggagtgatcatgacaatagccttgattctcatggtaacttcagctactgagttcatccggaggagttattttgaagtcttctggtatactcaccacctttttatcttctatatccttggcttagggattcacggcattggtggaattgtccggggtcaaacagaggagagcatgaatgagagtcatcctcgcaagtgtgcagagtcttttgagatgtgggatgatcgtgactcccactgtaggcgccctaagtttgaagggcatccccctgagtcttggaagtggatccttgcaccggtcattctttatatctgtgaaaggatcctccggttttaccgctcccagcagaaggttgtgattaccaaggttgttatgcacccatccaaagttttggaattgcagatgaacaagcgtggcttcagcatggaagtggggcagtatatctttgttaattgcccctcaatctctctcctggaatggcatccttttactttgacctctgctccagaggaagatttcttctccattcatatccgagcagcaggggactggacagaaaatctcataagggctttcgaacaacaatattcaccaattcccaggattgaagtggatggtccctttggcacagccagtgaggatgttttccagtatgaagtggctgtgctggttggagcaggaattggggtcaccccctttgcttctatcttgaaatccatctggtacaaattccagtgtgcagaccacaacctcaaaacaaaaaagatctatttctactggatctgcagggagacaggtgccttttcctggttcaacaacctgttgacttccctggaacaggagatggaggaattaggcaaagtgggttttctaaactaccgtctcttcctcaccggatgggacagcaatattgttggtcatgcagcattaaactttgacaaggccactgacatcgtgacaggtctgaaacagaaaacctcctttgggagaccaatgtgggacaatgagttttctacaatagctacctcccaccccaagtctgtagtgggagttttcttatgtggccctcggactttggcaaagagcctgcgcaaatgctgtcaccgatattccagtctggatcctagaaaggttcaattctacttcaacaaagaaaattttgagcagaagctcatctcagaagaagacctctga

SEQ ID NO: 33 - protein, 724 aa, Artificial sequence
  an artificially synthesized sequence
MYQRMLRCGAELGSPGGGSSGGAGGRLALLWIVPLTLSGLLGVAWGASSLGAHHIHHKLDYKDDDDKGGLANPTRVGGREPYPGSAEVIRESSSTTGMVVGIVAAAALCILILLYAMKKRRAKGGSGGSGGLEHGTIPFNRTHRSKRSSGMGNWVVNHWFSVLFLVVWLGLNVFLFVDAFLKYEKADKYYYTRKILGSTLACARASALCLNFNSTLILLPVCRNLLSFLRGTCSFCSRTLRKQLDHNLTFHKLVAYMICLHTAIHIIAHLFNFDCYSRSRQATDGSLASILSSLSHDEKKGGSWLNPIQSRNTTVEYVTFTSIAGLTGVIMTIALILMVTSATEFIRRSYFEVFWYTHHLFIFYILGLGIHGIGGIVRGQTEESMNESHPRKCAESFEMWDDRDSHCRRPKFEGHPPESWKWILAPVILYICERILRFYRSQQKVVITKVVMHPSKVLELQMNKRGFSMEVGQYIFVNCPSISLLEWHPFTLTSAPEEDFFSIHIRAAGDWTENLIRAFEQQYSPIPRIEVDGPFGTASEDVFQYEVAVLVGAGIGVTPFASILKSIWYKFQCADHNLKTKKIYFYWICRETGAFSWFNNLLTSLEQEMEELGKVGFLNYRLFLTGWDSNIVGHAALNFDKATDIVTGLKQKTSFGRPMWDNEFSTIATSHPKSVVGVFLCGPRTLAKSLRKCCHRYSSLDPRKVQFYFNKENFEQKLISEEDL

SEQ ID NO: 34 - protein, 592 aa, Artificial sequence
  an artificially synthesized sequence
MGNWVVNHWFSVLFLVVWLGLNVFLFVDAFLKYEKADKYYYTRKILGSTLACARASALCLNFNSTLILLPVCRNLLSFLRGTCSFCSRTLRKQLDHNLTFHKLVAYMICLHTAIHIIAHLFNFDCYSRSRQATDGSLASILSSLSHDEKKGGSWLNPIQSRNTTVEYVTFTSIAGLTGVIMTIALILMVTSATEFIRRSYFEVFWYTHHLFIFYILGLGIHGIGGIVRGQTEESMNESHPRKCAESFEMWDDRDSHCRRPKFEGHPPESWKWILAPVILYICERILRFYRSQQKVVITKVVMHPSKVLELQMNKRGFSMEVGQYIFVNCPSISLLEWHPFTLTSAPEEDFFSIHIRAAGDWTENLIRAFEQQYSPIPRIEVDGPFGTASEDVFQYEVAVLVGAGIGVTPFASILKSIWYKFQCADHNLKTKKIYFYWICRETGAFSWFNNLLTSLEQEMEELGKVGFLNYRLFLTGWDSNIVGHAALNFDKATDIVTGLKQKTSFGRPMWDNEFSTIATSHPKSVVGVFLCGPRTLAKSLRKCCHRYSSLDPRKVQFYFNKENFWSHPQFEKGGGSGGGSGGSAWSHPQFEK

SEQ ID NO: 35 - protein, 6 aa, Leporinae trouessart
SSYWIC

SEQ ID NO: 36 - protein, 18 aa, Leporinae trouessart
CINAGNSGSTYYASWAKG

SEQ ID NO: 37 - protein, 20 aa, Leporinae trouessart
GSYSYGYVGYVYTTSTRLDL

SEQ ID NO: 38 - protein, 11 aa, Leporinae trouessart
QASQNIGSDLA

SEQ ID NO: 39 - protein, 7 aa, Leporinae trouessart
RASTLAS

SEQ ID NO: 40 - protein, 13 aa, Leporinae trouessart
QCTYYGSIYVPNA

SEQ ID NO: 41 - protein, 129 aa, Leporinae trouessart
QSLEESGGDLVKPGASLTLTCTASGFSFSSSYWICWVRQAPGKGLEWIACINAGNSGSTYYASWAKGRFTISKTSSTTVTLQMTSLTAADTATYFCARGSYSYGYVGYVYTTSTRLDLWGQGTLVTVSS

SEQ ID NO: 42 - protein, 111 aa, Leporinae trouessart
DVVMTQTPASVSEPVGGTVTIKCQASQNIGSDLAWYQQKPGQPPKLLIYRASTLASGVSSRFKGSGSGTQFTLTISDLECADAATYYCQCTYYGSIYVPNAFGGGTEVVVK

SEQ ID NO: 43 - protein, 6 aa, Leporinae trouessart
DWYWVG

SEQ ID NO: 44 - protein, 17 aa, Leporinae trouessart
CIVTGNGHTYYASWAKG

SEQ ID NO: 45 - protein, 9 aa, Leporinae trouessart
GVGASRYAL

SEQ ID NO: 46 - protein, 12 aa, Leporinae trouessart
QASESVASNWLS

SEQ ID NO: 47 - protein, 7 aa, Leporinae trouessart
FASTLAS

SEQ ID NO: 48 - protein, 12 aa, Leporinae trouessart
AGYKSDTTDGTA

SEQ ID NO: 49 - protein, 117 aa, Leporinae trouessart
QSLEESGGDLVKPGGTLTLTCTASGFSFSDWYWVGWVRQAPGKGLEWIGCIVTGNGHTYYASWAKGRFTISKTSSTTVTLQLNSLTAADTATYFCARGVGASRYALWGPGTLVTVSS

SEQ ID NO: 50 - protein, 110 aa, Leporinae trouessart
IVMTQTPSSKSVPVGDTVTINCQASESVASNWLSWYQQKPGQPPKLLIYFASTLASGVPSRFKGSGSGTQFTLTISDVVCDDAATYYCAGYKSDTTDGTAFGGGTEVVVK

SEQ ID NO: 51 - protein, 5 aa, Leporinae trouessart
YYFMT

SEQ ID NO: 52 - protein, 16 aa, Leporinae trouessart
IIRVDGSGYYATWAKG

SEQ ID NO: 53 - protein, 8 aa, Leporinae trouessart
VGDWAFNI

SEQ ID NO: 54 - protein, 11 aa, Leporinae trouessart
QASEDIESYLA

SEQ ID NO: 55 - protein, 7 aa, Leporinae trouessart
RASTLAS

SEQ ID NO: 56 - protein, 12 aa, Leporinae trouessart
QQGHTINNIDNV

SEQ ID NO: 57 - protein, 113 aa, Leporinae trouessart
QSLEESGGRLVTPGTPLTLTCTASGFSLSYYFMTWVRQAPGKGLEYIGIIRVDGSGYYATWAKGRFTISRTSTTVELKITSPTTEDTATYFCARVGDWAFNIWGPGTLVTVSS

SEQ ID NO: 58 - protein, 109 aa, Leporinae trouessart
IKMTQTPASVSTAVGGTVSINCQASEDIESYLAWFQQKPGQRPKLLIYRASTLASGVSSRFKGSGSGTEFTLTISGVECADAATYYCQQGHTINNIDNVFGGGTEVVVK

SEQ ID NO: 59 - protein, 5 aa, Artificial sequence
  an artificially synthesized sequence
NAWMH

SEQ ID NO: 60 - protein, 19 aa, Artificial sequence
  an artificially synthesized sequence
QIKDKSQNYATYVAESVKG

SEQ ID NO: 61 - protein, 11 aa, Artificial sequence
  an artificially synthesized sequence
VHYAAGYGVDI

SEQ ID NO: 62 - protein, 16 aa, Artificial sequence
  an artificially synthesized sequence
RSSQPLVHSNRNTYLH

SEQ ID NO: 63 - protein, 7 aa, Artificial sequence
  an artificially synthesized sequence
KVSNRFS

SEQ ID NO: 64 - protein, 9 aa, Artificial sequence
  an artificially synthesized sequence
GQGTQVPYT

SEQ ID NO: 65 - protein, 122 aa, Artificial sequence
  an artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 66 - protein, 112 aa, Artificial sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 67 - protein, 6 aa, Leporinae trouessart
SNYFMC

SEQ ID NO: 68 - protein, 18 aa, Leporinae trouessart
CIYTGSSGGTYYATWAKG

SEQ ID NO: 69 - protein, 9 aa, Leporinae trouessart
DTMTIPFNL

SEQ ID NO: 70 - protein, 13 aa, Leporinae trouessart
QASQSVYNNNNLH

SEQ ID NO: 71 - protein, 7 aa, Leporinae trouessart
FASTLAS

SEQ ID NO: 72 - protein, 13 aa, Leporinae trouessart
QGEFSCSSADCNA

SEQ ID NO: 73 - protein, 119 aa, Leporinae trouessart
QEQLEESGGDLVKPGASLTLTCTASGFSFNSNYFMCWVRQAPGKGLEWIACIYTGSSGGTYYATWAKGRFTISKTSSTTVTLQMTSLTAADTATYFCAGDTMTIPFNLWGPGTLVTVSS

SEQ ID NO: 74 - protein, 112 aa, Leporinae trouessart
QVLTQTASPVSAPVGGTVTINCQASQSVYNNNNLHWFQQKPGQRPKLLIYFASTLASGVSSRFKGSGSGTQFTLTISGVQCDDAATYYCQGEFSCSSADCNAFGGGTEVVVK

SEQ ID NO: 75 - protein, 5 aa, Leporinae trouessart
NYAMS

SEQ ID NO: 76 - protein, 16 aa, Leporinae trouessart
IISSSGSTYYASWAKG

SEQ ID NO: 77 - protein, 10 aa, Leporinae trouessart
DLDASTGTDI

SEQ ID NO: 78 - protein, 13 aa, Leporinae trouessart
QASQSVYSNNYLS

SEQ ID NO: 79 - protein, 7 aa, Leporinae trouessart
KASTLAS

SEQ ID NO: 80 - protein, 12 aa, Leporinae trouessart
AGDYKSSSDIRA

SEQ ID NO: 81 - protein, 115 aa, Leporinae trouessart
QSVEESGGRLVTPGTPLTLTCTVSGFSLSNYAMSWVRQAPGKGLEWIGIISSSGSTYYASWAKGRFTISKTSTTVDLKITSPTTEDTATYFCARDLDASTGTDIWGPGTLVTVSS

SEQ ID NO: 82 - protein, 111 aa, Leporinae trouessart
AVLTQTPSPVSAAVGGTVTINCQASQSVYSNNYLSWFQQKPGQPPKLLIYKASTLASGVPSRFKGSGSGTQFTLTISDVQCDDAATYYCAGDYKSSSDIRAFGGGTEVVVK

SEQ ID NO: 83 - protein, 328 aa, Artificial sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 84 - protein, 328 aa, Artificial sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 85 - protein, 107 aa, Artificial sequence
  an artificially synthesized sequence
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 86 - protein, 107 aa, Artificial sequence [sequence omitted]

(19) United States
(12) Patent Application Publication
(10) Pub. No.: US 2022/0089770 A1
(43) Pub. Date: Mar. 24, 2022

(54) NOVEL CANCER ANTIGENS AND ANTIBODIES OF SAID ANTIGENS

(71) Applicant: Chugai Seiyaku Kabushiki Kaisha, Tokyo (JP)

(72) Inventors: Junichi NEZU, Kanagawa (JP); Tatsushi KODAMA, Singapore (SG); Mayumi HOSHINO, Kanagawa (JP); Naoki KIMURA, Kanagawa (JP); Kohji NAGANO, Kanagawa (JP); Kuniyasu KATO, Kanagawa (JP); Yukari YOKOTA, Kanagawa (JP)

(21) Appl. No.: 17/424,603

(22) PCT Filed: Jan. 24, 2020
(86) PCT No.: PCT/JP2020/002476

§ 371 Date: Jul. 21, 2021

(30) Foreign Application Priority Data
Jan. 24, 2019 (JP) 2019-009845

(51) Int. Cl. C07K 16/30; C07K 16/28; A61P 35/00

(52) U.S. Cl. CPC C07K 16/3023; C07K 16/2809; C07K 2317/73; C07K 2317/31; A61P 35/00

(57) ABSTRACT

In a non-limiting embodiment, isolated antibodies that bind to any of the following proteins are provided: XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4.

### TECHNICAL FIELD

The present disclosure relates to isolated antibodies that bind to or recognize any of the following proteins: XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4.

### BACKGROUND ART

Antibodies have received attention as drugs because of being highly stable in plasma and causing little side effects. Among them, many IgG-type antibody drugs have been launched, and a large number of antibody drugs are currently under development (NPLs 1 and 2).

Rituxan against CD20, cetuximab against EGFR, Herceptin against HER2, and the like have been approved so far as therapeutic drugs for cancer using antibody drugs (NPL 3). These antibody molecules bind to their antigens expressed on cancer cells and thereby exert cytotoxic activity against the cancer cells through ADCC activity, etc. Such cytotoxic activity based on ADCC activity, etc. is known to depend on the number of antigens expressed on target cells of therapeutic antibodies (NPL 4). Therefore, high expression levels of targeted antigens are preferred from the viewpoint of the effects of therapeutic antibodies. However, if an antigen, albeit having a high expression level, is expressed in normal tissues, the cytotoxic activity based on ADCC activity, etc. is exerted against the normal cells. Hence, side effects become a serious problem. Therefore, it is preferred that antigens targeted by therapeutic antibodies as therapeutic drugs for cancer should be expressed specifically on cancer cells. For example, an antibody molecule against EpCAM known as a cancer antigen had been considered promising as a therapeutic drug for cancer. However, the EpCAM is known to be also expressed in the pancreas. In actuality, it has been reported in clinical trials that the administration of an anti-EpCAM antibody causes pancreatitis as a side effect due to cytotoxic activity against the pancreas (NPL 5).

In the wake of the success of antibody drugs exerting cytotoxic activity based on ADCC activity, second-generation improved antibody molecules exerting strong cytotoxic activity have been reported as a result of, for example, enhancing ADCC activity by the removal of fucose from the N-linked oligosaccharide of a native human IgG1 Fc region (NPL 6) or enhancing ADCC activity by enhancing binding to FcγRIIIa through the amino acid substitution of a native human IgG1 Fc region (NPL 7). Improved antibody molecules exerting stronger cytotoxic activity, such as an antibody drug conjugate (ADC) containing an antibody conjugated with a drug having strong cytotoxic activity (NPL 8), and a low-molecular antibody exerting cytotoxic activity against cancer cells by recruiting T cells to the cancer cells (NPL 9) have also been reported as antibody drugs exerting cytotoxic activity against cancer cells under a mechanism other than NK cell-mediated ADCC activity as mentioned above.

Such antibody molecules exerting stronger cytotoxic activity can exert cytotoxic activity even against cancer cells expressing an antigen at a level that is not high, but also exert cytotoxic activity against normal tissues expressing the antigen at a low level, similarly to cancer cells. In actuality, EGFR-BiTE, a bispecific antibody against CD3 and EGFR, can exert strong cytotoxic activity against cancer cells and exert an antitumor effect, by recruiting T cells to the cancer cells, as compared with cetuximab, native human IgG1 against the EGFR. On the other hand, it has also been found that serious side effects appear by the administration of EGFR-BiTE to cynomolgus monkeys, because EGFR is also expressed in normal tissues (NPL 10). Also, ADC bivatuzumab mertansine containing mertansine conjugated with an antibody against CD44v6 highly expressed on cancer cells has been clinically found to cause severe dermal toxicity and hepatoxicity, because CD44v6 is also expressed in normal tissues (NPL 11).

As mentioned above, use of an antibody that can exert strong cytotoxic activity even against cancer cells expressing an antigen at low levels requires the target antigen to be expressed in an exceedingly cancer-specific manner. However, considering that a target antigen HER2 of Herceptin or a target antigen EGFR of cetuximab is also expressed in normal tissues, only a limited number of cancer antigens may be expressed in an exceedingly cancer-specific manner. Therefore, side effects ascribable to a cytotoxic effect on normal tissues may become a problem, though cytotoxic activity against cancer can be enhanced.

Various techniques have been developed as techniques applicable to second-generation antibody drugs. For example, techniques of improving effector functions, antigen-binding ability, pharmacokinetics, or stability or reducing a risk of immunogenicity have been reported (NPL 12). However, in order to solve the above-mentioned side effects and allow antibody drugs to act specifically on target cancer tissues, there is still a need to identify antigens that show almost no or little expression in normal tissues and are specifically expressed in cancer cells.

### CITATION LIST

### Non-Patent Literature

[NPL 1] Monoclonal antibody successes in the clinic. Janice M Reichert, Clark J Rosensweig, Laura B Faden & Matthew C Dewitz, Nat. Biotechnol. (2005) 23, 1073-1078
[NPL 2] The therapeutic antibodies market to 2008. Pavlou A K, Belsey M J., Eur. J. Pharm. Biopharm. (2005) 59 (3), 389-396
[NPL 3] Monoclonal antibodies: versatile platforms for cancer immunotherapy. Weiner L M, Surana R, Wang S., Nat. Rev. Immunol. (2010) 10 (5), 317-327
[NPL 4] Differential responses of human tumor cell lines to anti-p185HER2 monoclonal antibodies. Lewis G D, Figari I, Fendly B, Wong W L, Carter P, Gorman C, Shepard H M, Cancer Immunol Immunotherapy (1993) 37, 255-263
[NPL 5] ING-1, a monoclonal antibody targeting Ep-CAM in patients with advanced adenocarcinomas. de Bono J S, Tolcher A W, Forero A, Vanhove G F, Takimoto C, Bauer R J, Hammond L A, Patnaik A, White M L, Shen S, Khazaeli M B, Rowinsky E K, LoBuglio A F, Clin. Cancer Res. (2004) 10 (22), 7555-7565
[NPL 6] Non-fucosylated therapeutic antibodies as next-generation therapeutic antibodies. Satoh M, Iida S, Shitara K., Expert Opin. Biol. Ther. (2006) 6 (11), 1161-1173
[NPL 7] Optimizing engagement of the immune system by anti-tumor antibodies: an engineer's perspective. Desjarlais J R, Lazar G A, Zhukovsky E A, Chu S Y., Drug Discov. Today (2007) 12 (21-22), 898-910
[NPL 8] Antibody-drug conjugates: targeted drug delivery for cancer. Alley S C, Okeley N M, Senter P D., Curr. Opin. Chem. Biol. (2010) 14 (4), 529-537
[NPL 9] BiTE: Teaching antibodies to engage T-cells for cancer therapy. Baeuerle P A, Kufer P, Bargou R., Curr. Opin. Mol. Ther. (2009) 11 (1), 22-30
[NPL 10] T cell-engaging BiTE antibodies specific for EGFR potently eliminate KRAS- and BRAF-mutated colorectal cancer cells. Lutterbuese R, Raum T, Kischel R, Hoffmann P, Mangold S, Rattel B, Friedrich M, Thomas O, Lorenczewski G, Rau D, Schaller E, Herrmann I, Wolf A, Urbig T, Baeuerle P A, Kufer P., Proc. Natl. Acad. Sci. U.S.A. (2010) 107 (28), 12605-12610
[NPL 11] Phase I trial with the CD44v6-targeting immunoconjugate bivatuzumab mertansine in head and neck squamous cell carcinoma. Riechelmann H, Sauter A, Golze W, Hanft G, Schroen C, Hoermann K, Erhardt T, Gronau S., Oral Oncol. (2008) 44 (9), 823-829
[NPL 12] Antibody engineering for the development of therapeutic antibodies. Kim S J, Park Y, Hong H J., Mol. Cells. (2005) 20 (1), 17-29

### SUMMARY OF INVENTION

### Technical Problem

The invention in the present disclosure was achieved in view of the above circumstances. In a non-limiting embodiment, an objective of this invention is to provide antibodies that bind to or recognize an antigen that shows almost no or little expression in normal tissues and is highly expressed specifically in cancer cells.

### Solution to Problem

In a non-limiting embodiment, the present inventors conducted dedicated research and as a result discovered XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4 as cell surface proteins expressed at high levels in cancer tissues and at low levels in adjacent normal tissues and in normal tissues. Furthermore, the present inventors found that these proteins are highly cancer-specific antigens that are highly expressed in colorectal cancer tissues (particularly colorectal cancer tissues with a KRAS mutation) and/or lung cancer tissues.

The present disclosure is based on these findings, and specifically encompasses embodiments illustratively listed below:
[1] An isolated antibody that binds to any of the following proteins: XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4.
[2] The antibody of [1], which binds to an extracellular domain of the protein.
[3] The antibody of [1] or [2], which binds to any of:

(1) XPR1 represented by SEQ ID NO: 1,

(2) NOX1 represented by SEQ ID NO: 2,

(3) MARVELD3 isoform 1 represented by SEQ ID NO: 3,

(4) MARVELD3 isoform 2 represented by SEQ ID NO: 4,

(5) SPINT2 represented by SEQ ID NO: 5,

(6) MANSC1 represented by SEQ ID NO: 7,

(7) SLC12A2 represented by SEQ ID NO: 8,

(8) CDCP1 represented by SEQ ID NO: 9,

(9) SEZ6L2 represented by SEQ ID NO: 12,

(10) FLVCR1 represented by SEQ ID NO: 13,

(11) SLC7A5 represented by SEQ ID NO: 14,

(12) STEAP1 represented by SEQ ID NO: 15,

(13) MMP14 represented by SEQ ID NO: 16,

(14) TNFRSF21 represented by SEQ ID NO: 17, and

(15) TMPRSS4 represented by SEQ ID NO: 18.
[4] The antibody of [1] or [2], which binds to any of:
   (1) an extracellular domain of XPR1, which is any of amino acids 1 to 108, 111 to 122, 159 to 171, 177 to 216, 423 to 448, 473 to 502, 660 to 670, 674 to 696, 177 to 190, 428 to 448, 660 to 670, 244, 256 to 273, 257 to 270, 258 to 264, 258 to 268, 256 to 270, 258 to 273, 260 to 270, 293 to 314, 336 to 343, 337 to 344, 338 to 341, 340 to 342, 340 to 344, 343 to 344, 340 to 345, 368 to 372, 392 to 398, 397 to 401, 398 to 402, 420 to 442, 420 to 506, 465 to 479, 497 to 507, 498 to 508, 529 to 555, 529 to 570, 582 to 586, 1 to 234, 1 to 236, 293 to 318, 367 to 442, 369 to 473, 500 to 507, 529 to 696, and 589 to 696 in the amino acid sequence represented by SEQ ID NO: 1;
   (2) an extracellular domain of NOX1, which is any of amino acids 44 to 54, 131 to 161, 242 to 258, 1 to 4, 1 to 11, 18 to 55, 28 to 44, 31 to 44, 32 to 46, 34 to 50, 70 to 102, 70 to 103, 117 to 176, 120 to 172, 122 to 166, 122 to 172, 124 to 168, 190 to 208, 191 to 204, 223 to 266, 223 to 267, 227 to 269, 228 to 391, 228 to 396, 404, and 420 to 564 in the amino acid sequence represented by SEQ ID NO: 2;
   (3) an extracellular domain of MARVELD3 isoform 1, which is any of amino acids 222 to 266, 223 to 269, 227 to 264, 227 to 268, 229 to 263, 231 to 265, 321 to 362, 322 to 357, 323 to 357, 324 to 357, and 324 to 358 in the amino acid sequence represented by SEQ ID NO: 3; and
   (4) an extracellular domain of MARVELD3 isoform 2, which is any of amino acids 101 to 111, 163 to 198, 1 to 266, 1 to 270, 216 to 271, 222 to 269, 226 to 271, 227 to 268, 227 to 271, 248 to 271, 316 to 364, 322 to 360, 323 to 359, 324 to 360, 326 to 360, and 327 to 360 in the amino acid sequence represented by SEQ ID NO: 4.
[4A] The antibody of any one of [1], [2], [3](1), and [4](1), which can bind to XPR1, and is selected from the following:
   (a1) an antibody comprising HVR-H1 consisting of the amino acid sequence of SEQ ID NO: 35, HVR-H2 consisting of the amino acid sequence of SEQ ID NO: 36, HVR-H3 consisting of the amino acid sequence of SEQ ID NO: 37, HVR-L1 consisting of the amino acid sequence of SEQ ID NO: 38, HVR-L2 consisting of the amino acid sequence of SEQ ID NO: 39, and HVR-L3 consisting of the amino acid sequence of SEQ ID NO: 40;
   (a2) an antibody comprising the VH sequence of SEQ ID NO: 41 and the VL sequence of SEQ ID NO: 42;
   (a3) an antibody that binds to the same epitope in XPR1 as the antibody of any one of (a1) to (a2);
   (a4) an antibody that competes with the antibody of any one of (a1) to (a2) for binding to XPR1;
   (a5) an antibody that blocks the binding of the antibody of any one of (a1) to (a2) to XPR1 by 50% or more in a competitive assay; and
   (a6) an antibody comprising a VH sequence having at least 95% sequence identity with the amino acid sequence of SEQ ID NO: 41 and a VL sequence having at least 95% sequence identity with the amino acid sequence of SEQ ID NO: 42.
[4B] The antibody of any one of [1], [2], [3](2), and [4](2), which can bind to NOX1, and is selected from the following:
   (b1) an antibody comprising HVR-H1 consisting of the amino acid sequence of SEQ ID NO: 67, HVR-H2 consisting of the amino acid sequence of SEQ ID NO: 68, HVR-H3 consisting of the amino acid sequence of SEQ ID NO: 69, HVR-L1 consisting of the amino acid sequence of SEQ ID NO: 70, HVR-L2 consisting of the amino acid sequence of SEQ ID NO: 71, and HVR-L3 consisting of the amino acid sequence of SEQ ID NO: 72;
   (b2) an antibody comprising HVR-H1 consisting of the amino acid sequence of SEQ ID NO: 75, HVR-H2 consisting of the amino acid sequence of SEQ ID NO: 76, HVR-H3 consisting of the amino acid sequence of SEQ ID NO: 77, HVR-L1 consisting of the amino acid sequence of SEQ ID NO: 78, HVR-L2 consisting of the amino acid sequence of SEQ ID NO: 79, and HVR-L3 consisting of the amino acid sequence of SEQ ID NO: 80;
   (b3) an antibody comprising the VH sequence of SEQ ID NO: 73 and the VL sequence of SEQ ID NO: 74;
   (b4) an antibody comprising the VH sequence of SEQ ID NO: 81 and the VL sequence of SEQ ID NO: 82;
   (b5) an antibody that binds to the same epitope in NOX1 as the antibody of any one of (b1) to (b4);
   (b6) an antibody that competes with the antibody of any one of (b1) to (b4) for binding to NOX1;
   (b7) an antibody that blocks the binding of the antibody of any one of (b1) to (b4) to NOX1 by 50% or more in a competitive assay;
   (b8) an antibody comprising a VH sequence having at least 95% sequence identity with the amino acid sequence of SEQ ID NO: 73 and a VL sequence having at least 95% sequence identity with the amino acid sequence of SEQ ID NO: 74; and
   (b9) an antibody comprising a VH sequence having at least 95% sequence identity with the amino acid sequence of SEQ ID NO: 81 and a VL sequence having at least 95% sequence identity with the amino acid sequence of SEQ ID NO: 82.
[4C] The antibody of any one of [1], [2], [3](3), [3](4), [4](3), and [4](4), which can bind to MARVELD3 isoform 1 and/or MARVELD3 isoform 2, and is selected from the following:
   (c1) an antibody comprising HVR-H1 consisting of the amino acid sequence of SEQ ID NO: 43, HVR-H2 consisting of the amino acid sequence of SEQ ID NO: 44, HVR-H3 consisting of the amino acid sequence of SEQ ID NO: 45, HVR-L1 consisting of the amino acid sequence of SEQ ID NO: 46, HVR-L2 consisting of the amino acid sequence of SEQ ID NO: 47, and HVR-L3 consisting of the amino acid sequence of SEQ ID NO: 48;
   (c2) an antibody comprising HVR-H1 consisting of the amino acid sequence of SEQ ID NO: 51, HVR-H2 consisting of the amino acid sequence of SEQ ID NO: 52, HVR-H3 consisting of the amino acid sequence of SEQ ID NO: 53, HVR-L1 consisting of the amino acid sequence of SEQ ID NO: 54, HVR-L2 consisting of the amino acid sequence of SEQ ID NO: 55, and HVR-L3 consisting of the amino acid sequence of SEQ ID NO: 56;
   (c3) an antibody comprising the VH sequence of SEQ ID NO: 49 and the VL sequence of SEQ ID NO: 50;
   (c4) an antibody comprising the VH sequence of SEQ ID NO: 57 and the VL sequence of SEQ ID NO: 58;
   (c5) an antibody that binds to the same epitope in MARVELD3 isoform 1 and/or MARVELD3 isoform 2 as the antibody of any one of (c1) to (c4);
   (c6) an antibody that competes with the antibody of any one of (c1) to (c4) for binding to MARVELD3 isoform 1 and/or MARVELD3 isoform 2;
   (c7) an antibody that blocks the binding of the antibody of any one of (c1) to (c4) to MARVELD3 isoform 1 and/or MARVELD3 isoform 2 by 50% or more in a competitive assay;
   (c8) an antibody comprising a VH sequence having at least 95% sequence identity with the amino acid sequence of SEQ ID NO: 49 and a VL sequence having at least 95% sequence identity with the amino acid sequence of SEQ ID NO: 50; and
   (c9) an antibody comprising a VH sequence having at least 95% sequence identity with the amino acid sequence of SEQ ID NO: 57 and a VL sequence having at least 95% sequence identity with the amino acid sequence of SEQ ID NO: 58.
[5] The antibody of any one of [1] to [4C], which has cytotoxic activity.
[6] The antibody of any one of [1] to [5], which has internalization activity.
[7] The antibody of any one of [1] to [6], which is a monoclonal antibody.
[8] The antibody of any one of [1] to [7], which is a human antibody, a humanized antibody, or a chimeric antibody.
[9] The antibody of any one of [1] to [8], which is an antibody fragment.
[10] The antibody of any one of [1] to [8], which is a full-length IgG antibody.
[11] The antibody of any one of [1] to [10], which is a multispecific antibody.
[12] The antibody of [11], further comprising a T cell receptor complex-binding domain.
[13] The antibody of [11] or [12], wherein the multispecific antibody comprises one binding domain for the protein.
[14] The antibody of any one of [11] to [13], comprising an Fc region with reduced Fcγ receptor-binding activity.
[15] The antibody of [14], wherein the Fc region has lower Fcγ receptor-binding activity than the Fc region of IgG1, IgG2, IgG3, or IgG4.
[16] The antibody of [12] or [15], wherein the antibody has cytotoxic activity, and the cytotoxic activity is a T cell-dependent cytotoxic activity.
[17] The antibody of any one of [12] to [16], wherein the T cell receptor complex-binding domain is a T cell receptor-binding domain having T cell receptor-binding activity.
[18] The antibody of any one of [12] to [16], wherein the T cell receptor complex-binding domain is a CD3-binding domain having CD3-binding activity.
[19] The antibody of [18], wherein the CD3-binding domain can bind to a CD3c chain.
[20] The antibody of [18] or [19], wherein the CD3-binding domain comprises an antibody heavy chain variable region and an antibody light chain variable region.
[20A] The antibody of any one of [18] to [20], wherein the CD3-binding domain is selected from the following:
   (d1) a domain comprising an antibody variable region having HVR-H1 consisting of the amino acid sequence of SEQ ID NO: 59, HVR-H2 consisting of the amino acid sequence of SEQ ID NO: 60, HVR-H3 consisting of the amino acid sequence of SEQ ID NO: 61, HVR-L1 consisting of the amino acid sequence of SEQ ID NO: 62, HVR-L2 consisting of the amino acid sequence of SEQ ID NO: 63, and HVR-L3 consisting of the amino acid sequence of SEQ ID NO: 64;
   (d2) a domain comprising an antibody variable region having the VH sequence of SEQ ID NO: 65 and the VL sequence of SEQ ID NO: 66;
   (d3) a domain comprising an antibody variable region that binds to the same epitope in CD3 as the antibody variable region of any one of (d1) to (d2);
   (d4) a domain comprising an antibody variable region that competes with the antibody variable region of any one of (d1) to (d2) for binding to CD3;
   (d5) a domain comprising an antibody variable region that blocks the binding of the antibody variable region of any one of (d1) to (d2) to CD3 by 50% or more in a competitive assay; and
   (d6) a domain comprising an antibody variable region comprising a VH sequence having at least 95% sequence identity with the amino acid sequence of SEQ ID NO: 65 and a VL sequence having at least 95% sequence identity with the amino acid sequence of SEQ ID NO: 66.
[21] The antibody of any one of [11] to [20A], wherein the multispecific antibody is a bispecific antibody.
[22] An isolated nucleic acid encoding the antibody of any one of [1] to [21].
[23] A host cell comprising the nucleic acid of [22].
[24] A method for producing the antibody of any one of [1] to [21], comprising culturing the host cell of [23] such that the antibody is produced.
[25] The method of [24], further comprising recovering the antibody from the host cell.
[26] An immunoconjugate comprising the antibody of any one of [1] to [21] and a cytotoxic agent.
[27] A pharmaceutical formulation comprising the antibody of any one of [1] to [21] and a pharmaceutically acceptable carrier.
[28] The antibody of any one of [1] to [21] for use as a pharmaceutical.
[29] The antibody of any one of [1] to [21] for use in treatment of cancer.
[30] The antibody of [29], wherein the cancer is lung cancer, and the antibody binds to any of XPR1, SPINT2, SEZ6L2, FLVCR1, SLC7A5, STEAP1, MMP14, TNFRSF21, and TMPRSS4.
[31] The antibody of [30], wherein the lung cancer does not have one or more selected from an EGFR driver mutation, a BRAF driver mutation, an ERBB2 driver mutation, ALK fusion gene expression, and RET/ROS1 fusion gene expression.
[32] The antibody of [30] or [31], wherein the lung cancer is lung adenocarcinoma.
[33] The antibody of [29], wherein the cancer is colorectal cancer, and the antibody binds to any of NOX1, MARVELD3 isoform 1, MARVELD3 isoform 2, SPINT2, MANSC1, SLC12A2, and CDCP1.
[34] The antibody of [33], wherein the colorectal cancer has a KRAS mutation.
[35] The antibody of any one of [1] to [21], which is for use in inducing cytotoxic activity.
[36] Use of the antibody of any one of [1] to [21] in the manufacture of a pharmaceutical.
[37] Use of the antibody of any one of [1] to [21] in the manufacture of a pharmaceutical for cancer treatment.
[38] The use of [37], wherein the cancer is lung cancer, and the antibody binds to any of XPR1, SPINT2, SEZ6L2, FLVCR1, SLC7A5, STEAP1, MMP14, TNFRSF21, and TMPRSS4.
[39] The use of [38], wherein the lung cancer does not have one or more selected from an EGFR driver mutation, a BRAF driver mutation, an ERBB2 driver mutation, ALK fusion gene expression, and RET/ROS1 fusion gene expression.
[40] The use of [38] or [39], wherein the lung cancer is lung adenocarcinoma.
[41] The use of [37], wherein the cancer is colorectal cancer, and the antibody binds to any of NOX1, MARVELD3 isoform 1, MARVELD3 isoform 2, SPINT2, MANSC1, SLC12A2, and CDCP1.
[42] The use of [41], wherein the colorectal cancer has a KRAS mutation.
[43] Use of the antibody of any one of [1] to [21] in the manufacture of a pharmaceutical that induces cytotoxic activity.
[44] A method for treating an individual having cancer, comprising administering an effective amount of the antibody of any one of [1] to [21] to the individual.
[45] The method of [44], wherein the cancer is lung cancer, and the antibody binds to any of XPR1, SPINT2, SEZ6L2, FLVCR1, SLC7A5, STEAP1, MMP14, TNFRSF21, and TMPRSS4.
[46] The method of [45], wherein the lung cancer does not have one or more selected from an EGFR driver mutation, a BRAF driver mutation, an ERBB2 driver mutation, ALK fusion gene expression, and RET/ROS1 fusion gene expression.
[47] The method of [45] or [46], wherein the lung cancer is lung adenocarcinoma.
[48] The method of [44], wherein the cancer is colorectal cancer, and the antibody binds to any of NOX1, MARVELD3 isoform 1, MARVELD3 isoform 2, SPINT2, MANSC1, SLC12A2, and CDCP1.
[49] The method of [48], wherein the colorectal cancer has a KRAS mutation.
[50] A method for inducing cytotoxic activity in an individual, comprising administering an effective amount of the antibody of any one of [1] to [21] to the individual to induce cytotoxic activity.
[51] A method for detecting the presence of XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, or TMPRSS4 in a biological sample, which comprises contacting the antibody of any one of [1] to [21] that can bind to a protein to be detected, with the biological sample under conditions permissive for binding of the antibody to the protein to be detected.
[52] A method for detecting a cancer cell in a biological sample, which comprises contacting the antibody of any one of [1] to [21] with the biological sample under conditions permissive for binding of the antibody to a protein that serves as an antigen of the antibody.
[53] A method for detecting a colorectal cancer cell in a biological sample, which comprises contacting the antibody of any one of [1] to [21] that can bind to NOX1, MARVELD3 isoform 1, MARVELD3 isoform 2, SPINT2, MANSC1, SLC12A2, or CDCP1, with the biological sample under conditions permissive for binding of the antibody to a protein that serves as an antigen of the antibody.
[54] A method for detecting a colorectal cancer cell having a KRAS mutation in a biological sample, which comprises contacting the antibody of any one of [1] to [21] that can bind to NOX1, MARVELD3 isoform 1, MARVELD3 isoform 2, SPINT2, MANSC1, SLC12A2, or CDCP1, with the biological sample under conditions permissive for binding of the antibody to a protein that serves as an antigen of the antibody.
[55] A method of detecting a lung cancer cell in a biological sample, which comprises contacting the antibody of any one of [1] to [21] that can bind to XPR1, SPINT2, SEZ6L2, FLVCR1, SLC7A5, STEAP1, MMP14, TNFRSF21, or TMPRSS4, with the biological sample under conditions permissive for binding of the antibody to a protein that serves as an antigen of the antibody.
[56] A method for detecting in a biological sample a lung cancer cell that does not have one or more selected from an EGFR driver mutation, a BRAF driver mutation, an ERBB2 driver mutation, ALK fusion gene expression, and RET/ROS1 fusion gene expression, wherein the method comprises contacting the antibody of any one of [1] to [21] that can bind to XPR1, SPINT2, SEZ6L2, FLVCR1, SLC7A5, STEAP1, MMP14, TNFRSF21, or TMPRSS4, with the biological sample under conditions permissive for binding of the antibody to a protein that serves as an antigen of the antibody.

### BRIEF DESCRIPTION OF DRAWINGS

FIG. 1-1 shows the protein expression of each target antigen candidate. The vertical axis shows the name of the molecule and the quantification value of protein expression (iBAQ value), and the horizontal axis shows the analyzed tissues. All LC-MS data (N=3) of all samples from each tissue were plotted. Closed circles (●) indicate colorectal cancer tissue samples with a KRAS mutation or lung cancer tissue samples with a driver mutation. Open circles (∘) indicate samples without the above mutations.

FIG. 1-2: Refer to the description of FIG. 1-1.

FIG. 1-3: Refer to the description of FIG. 1-1.

FIG. 1-4: Refer to the description of FIG. 1-1.

FIG. 1-5: Refer to the description of FIG. 1-1.

FIG. 1-6: Refer to the description of FIG. 1-1.

FIG. 1-7: Refer to the description of FIG. 1-1.

FIG. 1-8: Refer to the description of FIG. 1-1.

FIG. 1-9: Refer to the description of FIG. 1-1.

FIG. 1-10: Refer to the description of FIG. 1-1.

FIG. 1-11: Refer to the description of FIG. 1-1.

FIG. 1-12: Refer to the description of FIG. 1-1.

FIG. 1-13: Refer to the description of FIG. 1-1.

FIG. 1-14: Refer to the description of FIG. 1-1.

FIG. 1-15: Refer to the description of FIG. 1-1.

FIG. 1-16: Refer to the description of FIG. 1-1.

FIG. 2 shows an alignment of the amino acid sequences of isoforms 1 and 2 of MARVELD3 (SEQ ID NOs: 3 and 4, respectively). Extracellular regions are underlined based on FIG. 1A of Raleigh D. R. et al., Mol. Biol. Cell 21, 2010.

FIG. 3-1: Comparison of the expression levels of MARVELD3 isoform 1- and 2-specific peptides in colorectal cancer samples. FIG. 3-1 shows the expression levels of MARVELD3 isoform 2, and FIG. 3-2 shows the expression levels of MARVELD3 isoform 1. Since EKPAEMFEF was identified as an isoform 2-specific peptide and QLDQQYTILR was identified as an isoform 1-specific peptide, the signal intensity of these peptides was used to compare the expression levels. The vertical axes show the signal intensity of the isoform 1- and 2-specific peptides, and the horizontal axes show the sample names of colorectal cancer tissues and adjacent normal tissues (each sample was analyzed by LC-MS at N=3). Black bars indicate samples with a KRAS mutation, and gray bars indicate samples without a KRAS mutation.

FIG. 3-2: Refer to the description of FIG. 3-1.

FIG. 4-1 shows the results of cell surface localization analyses of XPR1, NOX1, and MARVELD3 isoform 2 (SEQ ID NOs: 1, 2, and 4, respectively). In the full amino acid sequence of each protein, transmembrane regions are boxed with a single line and extracellular regions are boxed with a double line, according to the UniProt topology information. Dashed lines indicate peptides identified in overexpressing cells, and portions with black underlines and bold amino acid letters indicate peptides identified from the endogenous protein.

FIG. 4-2: Refer to the description of FIG. 4-1.

FIG. 4-3: Refer to the description of FIG. 4-1.

FIG. 5-1 shows the expression levels of XPR1 in NCI-H2227 cells and HLC-1 cells.

FIG. 5-2 shows the expression level of MARVELD3 isoform2 in Caco-2 cells.

FIG. 6 shows the Alexa488 staining of a fraction stained with CellTrace FarRed (cells transfected with an empty plasmid vector) and a fraction not stained with CellTrace FarRed (cells transfected with the human XPR1-Myc expression plasmid vector).

FIG. 7 shows the cell growth inhibition rate of an anti-human XPR1/anti-human CD3 bispecific antibody.

FIG. 8 shows the cell growth inhibition rate of an anti-human XPR1/anti-human CD3 bispecific antibody.

FIG. 9 shows the Alexa488 staining of a fraction stained with CellTrace FarRed (cells transfected with an empty plasmid vector) and a fraction not stained with CellTrace FarRed (cells transfected with a human MARVELD3 isoform2-expressing plasmid vector).

FIG. 10 shows the cell growth inhibition rate of an anti-human MARVELD3 isoform2/anti-human CD3 bispecific antibody.

FIG. 11 shows the Alexa488 staining of a fraction not stained with CellTrace FarRed and CellTrace Violet (cells transfected with human NOX1-Strep), a fraction stained with CellTrace FarRed and CellTrace Violet (cells transfected with NOX1_Nx1B_564-myc), and a fraction stained only with CellTrace FarRed (cells transfected with an empty plasmid vector).

### DESCRIPTION OF EMBODIMENTS

### I. Definitions

The terms “anti-target protein antibody” and “an antibody that binds to a target protein” refer to an antibody that is capable of binding a target protein with sufficient affinity such that the antibody is useful as a diagnostic and/or therapeutic agent in targeting the target protein. In one embodiment, the extent of binding of an anti-target protein antibody to an unrelated, non-target protein is less than about 10% of the binding of the antibody to the target protein as measured, e.g., by a radioimmunoassay (RIA). In certain embodiments, an antibody that binds to a target protein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹M to 10⁻¹³ M). In certain embodiments, an anti-target protein antibody binds to an epitope of the target protein that is conserved among the target proteins from different species.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “chimeric” antibody refers to an antibody in which a portion of the heavy and/or light chain is derived from a particular source or species, while the remainder of the heavy and/or light chain is derived from a different source or species.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

In an exemplary competition assay, an immobilized target antigen (XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, or TMPRSS4) is incubated in a solution comprising a first labeled antibody that binds to the target protein (e.g., XPB0062, NXA0125, NXA0164, MDA0279, or MDA0314) and a second unlabeled antibody that is being tested for its ability to compete with the first antibody for binding to the target protein. The second antibody may be present in a hybridoma supernatant. As a control, the immobilized target protein is incubated in a solution comprising the first labeled antibody but not the second unlabeled antibody. After incubation under conditions permissive for binding of the first antibody to the target protein, excess unbound antibody is removed, and the amount of label associated with the immobilized target protein is measured. If the amount of label associated with the immobilized target protein is substantially reduced in the test sample relative to the control sample, then that indicates that the second antibody is competing with the first antibody for binding to the target protein. See Harlow and Lane (1988) Antibodies: A Laboratory Manual ch. 14 (Cold Spring Harbor Laboratory, Cold Spring Harbor, N.Y.).

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

### Antibody-Dependent Cell-Mediated Cytotoxicity

“Antibody-dependent cell-mediated cytotoxicity” or “ADCC” refers to a form of cytotoxicity in which the antibody Fc region bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### Human Effector Cells

“Human effector cells” refer to leukocytes that express one or more FcRs and perform effector functions. In certain embodiments, the cells express at least Fc gamma RIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

When a conjugate of the antibody of the present disclosure with a drug is incorporated into a cell, the conjugate-linked growth inhibitor or cytotoxic substance such as a toxic peptide can induce cell death of the cell that incorporated this antibody. Therefore, the antibody to which the growth inhibitor or the cytotoxic substance such as a toxic peptide is linked preferably also has internalization activity. In the present disclosure, “antibody having internalization activity” refers to an antibody that is transported into a cell (into the cytoplasm, vesicles, other organelles, and such) upon binding to a target protein on the cell surface. Whether or not an antibody has internalization activity can be confirmed using methods known to those skilled in the art. For example, the internalization activity can be confirmed by the method of contacting an antibody linked to a labeled substance with cells expressing its target protein and determining whether the labeled substance is incorporated into the cells, or the method of contacting an antibody linked to a growth inhibitor or a cytotoxic substance such as a toxic peptide with cells expressing its target protein and determining whether cell death is induced in the target protein-expressing cells.

An “immunoconjugate” is an antibody conjugated to one or more heterologous molecule(s), including but not limited to a cytotoxic agent.

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “naked antibody” refers to an antibody that is not conjugated to a heterologous moiety (e.g., a cytotoxic moiety) or radiolabel. The naked antibody may be present in a pharmaceutical formulation.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “effective amount” of an agent, e.g., a pharmaceutical formulation, refers to an amount effective, at dosages and for periods of time necessary, to achieve the desired therapeutic or prophylactic result.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the present disclosure are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### Cancer

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### Tumor

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

Herein, the term “driver mutation” means a gene mutation that directly causes the occurrence or malignant transformation of cancer. For example, important driver mutations in lung cancer and colorectal cancer include gene mutations in ALK, RET/ROS1, KRAS, EGFR, BRAF, and ERBB2. Non-limiting examples of driver mutations include, for example, the mutations listed in Table 2.

In one embodiment, the antibodies of the present disclosure are useful for at least one of treatment, prevention, and diagnosis of lung cancer. In certain embodiments, the antibodies of the present disclosure are useful for at least one of treatment, prevention, and diagnosis of lung cancer that does not have one or more selected from an EGFR driver mutation, a BRAF driver mutation, an ERBB2 driver mutation, ALK fusion gene expression, and RET/ROS1 fusion gene expression. In another embodiment, the antibodies of the present disclosure are useful in at least one of treatment, prevention, and diagnosis of colorectal cancer. In certain embodiments, the antibodies of the present disclosure are useful for at least one of treatment, prevention, and diagnosis of colorectal cancer with a KRAS mutation.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding” an antibody of the present disclosure refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### II. Compositions and Methods

In one aspect, the present disclosure is based, in part, on the discovery of proteins that are highly expressed specifically in cancer such as lung cancer and colorectal cancer. In certain embodiments, antibodies that bind to any of the following target proteins are provided: XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4. Antibodies of the present disclosure are useful, e.g., for the diagnosis or treatment of cancer such as lung cancer and colorectal cancer.

### Exemplary Target Proteins

The term “target protein,” as used herein, refers to any native target protein from any vertebrate source, including mammals such as primates (e.g. humans) and rodents (e.g., mice and rats), unless otherwise indicated. The term encompasses a “full-length” unprocessed target protein as well as any form of target protein that results from processing in the cell. The term also encompasses naturally occurring variants of the target protein, e.g., splice variants or allelic variants. The amino acid sequences of exemplary target proteins of the present disclosure are shown in SEQ ID NO: 1 (XPR1), SEQ ID NO: 2 (NOX1), SEQ ID NO: 3 (MARVELD3 isoform 1), SEQ ID NO: 4 (MARVELD3 isoform 2), SEQ ID NO: 5 (SPINT2), SEQ ID NO: 7 (MANSC1), SEQ ID NO: 8 (SLC12A2), SEQ ID NO: 9 (CDCP1), SEQ ID NO: 12 (SEZ6L2), SEQ ID NO: 13 (FLVCR1), SEQ ID NO: 14 (SLC7A5), SEQ ID NO: 15 (STEAP1), SEQ ID NO: 16 (MMP14), SEQ ID NO: 17 (TNFRSF21), and SEQ ID NO: 18 (TMPRSS4). Explanations of exemplary target proteins of the present disclosure are provided below.

### XPR1

XPR1 (xenotropic and polytropic retrovirus receptor) is a receptor for xenotropic/polytropic mouse leukemia viruses (X/P-MLVs) to infect and enter into cells. XPR1 consists of 696 amino acids, and has six asparagine-linked glycosylation sequences, eight cellular transmembrane domains, and four extracellular loops in this sequence (Proc Natl Acad Sci USA. 1999, 96:927-932. doi: 10.1073/pnas.96.3.927; Proc Natl Acad Sci USA. 1999, 96:1385-1390. doi: 10.1073/pnas.96.4.1385; and Nat Genet. 1999, 21:216-219). The inherent function of this protein in cells is unknown. No molecule highly homologous to XPR1 has been found in mammals, but in yeast, the SYG1 protein (J Biol Chem. 1995, 270:25435-25444), which is involved in the G protein-coupled division signaling, has been found to have a certain homology. The N-terminal hydrophilic region of XPR1 shows homology, although low, with NUC-2S of Neurospora crassa, and PHO81 and PHO85 of S. cerevisiae (Trends Genet. 1995, 11:209-211; Trends Biochem Sci. 1996, 21:383-387; and Mol Gen Genet. 1996, 252:709-716). Since these molecules are involved in the regulation of phosphate transport, it is considered that XPR1 may be involved in signal transduction and control of phosphate transport (Proc Natl Acad Sci USA. 1999, 96:1385-1390. doi: 10.1073/pnas.96.4.1385). In addition, it has been reported that RANKL stimulation increases the expression of XPR1 mRNA in bone marrow cells and macrophages, suggesting that the expression is regulated by the RANKL-RANK signal (Biochem Biophys Res Commun. 2010 Aug. 20, 399(2):129-32).

XPR1 has four extracellular loops (ECL1-4), and ECL3 and ECL4 are especially important as receptors for virus entry. Amino acid mutations in these regions are known to influence differences in susceptibility to virus infection (J Virol. 2007 October, 81(19):10550-7; Retrovirology. 2009 Oct. 7, 6( ):87; J Virol. 2010 November, 84(22):11970-80; J Virol. 1999 November, 73 (11):9362-8; Retrovirology. 2005 Dec. 15, 2( ):76; Retrovirology. 2010 Nov. 30, 7:101; and Virology. 2016 October, 497: 53-58).

There have been no reports on the expression and function of XPR1 in cancer.

XPR1 [NP_004727.2] (SEQ ID NO: 1) consists of 696 amino acids. Based on analyses by various topology prediction programs (PSORT II, UniProt, Phobius, and PolyPhobius) and article information (Proc Natl Acad Sci USA. 1999, 96:927-932. doi: 10.1073/pnas.96.3.927; Proc Natl Acad Sci USA. 1999, 96:1385-1390. doi: 10.1073/pnas.96.4.1385; and Nat Genet. 1999, 21:216-219), the following amino acid regions may be epitopes for XPR1 isoform 1-recognizing TRAB antibodies: 244, 256-273, 257-270, 258-264, 258-268, 258-273, 260-270, 293-314, 336-343, 337-344, 338-341, 340-342, 340-344, 340-345, 368-372, 392-398, 397-401, 398-402, 420-442, 420-506, 465-479, 497-507, 498-508, 529-555, 529-570, and 582-586. On the other hand, two topology prediction programs (TMHMM and Tmpred) predict extracellular regions very differently, and the following amino acid regions may be epitopes for XPR1 isoform 1-recognizing TRAB antibodies: amino acid regions 1-234, 1-236, 293-318, 367-442, 369-473, 500-507, 529-696, and 589-696.

### NOX1 (NADPH Oxidase Homolog 1, NOH1, Mitogenic Oxidase 1, MOX1, and GP91-2)

NADPH oxidase is a membrane-bound enzyme complex responsible for ROS production and is comprised of NOX family proteins which serve as catalytic domains and several other proteins. The NOX family is known to have seven family members, namely NOX 1 to 5 and DUOX1 and 2, and NOX1 is one of the family members (Physiol Rev. 2007, 87, 245-313; and Cell Mol. Life Sci. 2012, 69, 2327-2343).

ROS is generally considered to be involved in the development of various chronic diseases (arteriosclerosis, hypertension, and inflammation) by acting as a cytotoxic or mutagenic substance and damaging cells (Free Radical Biol. Med., 2007, 43, 332-347; and Cancer Sci., 2009, 100 (8), 1382-1388). On the other hand, elevated ROS levels have been observed in cancer, and ROS is considered to play an important role in the progression of cancer (Cancer. Lett., 2008, 266 (1), 37-52). In fact, an increase in NOX/DUOX expression has been observed in many cancer types (Anticancer Agents Med Chem. 2013, 13(3):502-14; and Clinical Science, 2015, 128, 863-875).

Increased expression of NOX1 was also observed in colorectal cancer, and in particular, K-Ras mutation has been reported to correspond with NOX1 expression (Int J Cancer. 2008, 123(1):100-7). KRas mutation-mediated activation of downstream signals induces senescence, and ROS produced by NOX1 reportedly plays an important role in inducing senescence (Genes Cells. 2013 January; 18 (1):32-41).

NOX1 [NP_008983.2] (SEQ ID NO: 2) consists of 546 amino acids. Based on analyses by various topology prediction programs (PSORT II, UniProt, TMHMM, Tmpred, Phobius, and PolyPhobius) and article information (Gene. 2001, 16, 269(1-2):131-40; and Biochem Biophys Res Commun. 2014, 17; 443(3):1060-5), the following amino acid regions may be epitopes for NOX1-recognizing TRAB antibodies: 1-4, 1-11, 18-55, 28-44, 31-44, 32-46, 34-50, 70-102, 70-103, 117-176, 120-172, 122-166, 122-172, 124-168, 190-208, 191-204, 223-266, 223-267, 227-269, 228-391, 228-396, 404, and 420-564.

MARVELD3 (MarvelD3, Marveld3, and marveld3)

The intercellular adhesion surface of epithelial cells and endothelial cells is called the tight junction (TJ) and is comprised of four different proteins: claudin, occludin, tricellulin, and marvelD3. Among them, occludin, tricellulin, and marvelD3 have a marvel domain and are therefore called the TJ-associated marvel protein (TAMP) family. These TJ proteins are all considered to be four-transmembrane proteins having intracellular N-terminal and C-terminal domains and two extracellular loops (Mol. Biol. Cell. 2010, 21, 1200-1213. doi:10.1091/mbc.E09-08-0734).

MarvelD3 has two isoforms (BMC Cell Biol. 2009, Dec. 22, 10:95. doi: 10). Isoform 1 [NP_001017967.2] (SEQ ID NO: 3) encodes 410 amino acids, and isoform 2 [NP_443090.4] (SEQ ID NO: 4) encodes 401 amino acids. Amino acid sequence-based structure prediction using TMPRED also presumes that marvelD3 is, like other TJ proteins, a protein with intracellular N- and C-termini, four transmembrane domains, and two extracellular loops (http://www.ch.embnet.org/software/TMPRED_form.html).

In addition, comparison of the amino acid sequences of the two isoforms shows that both share common N-terminal 198 amino acids, but the subsequent C-terminal sequence is completely different between the two isoforms (BMC Cell Biol. 2009, Dec. 22, 10:95. doi: 10). Expression of isoforms 1 and 2 at the gene level has been observed in cultured cells and various organs, but expression at the protein level has not been analyzed (BMC Cell Biol. 2009, Dec. 22, 10:95. doi: 10). Furthermore, functional differences and such between the two isoforms are not known.

MarvelD3 has been shown to colocalize with occludin at the cell junction in both endogenous expression cell lines and forced expression cell lines (BMC Cell Biol. 2009, Dec. 22, 10:95. doi: 10). Furthermore, on the cell membrane, marvelD3 has been shown to bind homophilically in cis and also heterophilically bind to both occludin and tricellulin in cis, indicating that marvelD3 is involved in TJ formation by interacting with other TAMPs (J Cell Sci 2013, 126: 554-564).

Little is known about the role of marvelD3 in cancer, except that its expression is reportedly reduced in undifferentiated pancreatic cancer cells and pancreatic cancer cells in which EMT is induced by snail (Exp Cell Res. 2011, Oct. 1, 317(16): 2288-98).

Based on analyses by various topology prediction programs (PSORT II, UniProt, TMHMM, Tmpred, Phobius, and PolyPhobius) and article information (Mol. Biol. Cell. 2010, 21, 1200-1213. doi:10.1091/mbc.E09-08-0734; and BMC Cell Biol. 2009, Dec. 22, 10:95. doi: 10), the following amino acid regions may be epitopes for MARVELD3 isoform 1-recognizing TRAB antibodies: 222-266, 223-269, 227-264, 227-268, 229-263, 231-265, 321-362, 322-357, 323-357, 324-357, and 324-358. Similarly, the following amino acid regions may be epitopes for MARVELD3 isoform 2-recognizing TRAB antibodies: 1-266, 1-270, 216-271, 222-269, 226-271, 227-268, 227-271, 248-271, 316-364, 322-360, 323-359, 324-360, 326-360, and 327-360. Meanwhile, some prediction programs give analysis results with three transmembrane domains, in contrast to what is mentioned above.

### SPINT2

SPINT2 (Kunitz-type protease inhibitor 2) [NP_066925.1] (SEQ ID NO: 5) is a transmembrane protein having two extracellular Kunitz domains that inhibit serine proteases (https://www.ncbi.nlm.nih.gov/protein/NP_066925.1).

### MANSC1

MANSC1 (MANSC domain-containing protein 1) is a protein represented by [NP_060520.2] (SEQ ID NO: 7) (https://www.ncbi.nlm.nih.gov/protein/NP_060520.2).

### SLC12A2

SLC12A2 (solute carrier family 12 member 2) [NP_001037.1] (SEQ ID NO: 8) is a membrane protein involved in the secretion and uptake of sodium and chlorides, and is said to have an important role in regulating ionic balance and cell volume (https://www.ncbi.nlm.nih.gov/protein/NP_001037.1).

### CDCP1

CDCP1 (CUB domain-containing protein 1) [NP_073753.3] (SEQ ID NO: 9) is a transmembrane protein having three extracellular domains and is known to be phosphorylated by Src family kinases (https://www.ncbi.nlm.nih.gov/protein/NP_073753.3).

### SEZ6L2

SEZ6L2 (Seizure 6-like protein 2) [NP_963869.2] (SEQ ID NO: 12) is a protein localized in the cell membrane, and is said to be possibly involved in endoplasmic reticulum function in nerve cells (https://www.uniprot.org/uniprot/Q6UXD5; and https://www.ncbi.nlm.nih.gov/protein/NP_963869.2).

### FLVCR1

FLVCR1 (feline leukemia virus subgroup C receptor-related protein 1) [NP_054772.1] (SEQ ID NO: 13) is a heme transporter and is considered to play an important role in the formation of red blood cells (https://www.ncbi.nlm.nih.gov/protein/NP_054772.1).

### SLC7A5

SLC7A5 (solute carrier family 7 member 5) is a protein represented by [NP_003477.4] (SEQ ID NO: 14) and is also called large neutral amino acids transporter small subunit 1 (https://www.ncbi.nlm.nih.gov/protein/NP_003477.4).

### STEAP1

STEAP1 (metalloreductase STEAP1) is a protein represented by [NP_036581.1] (SEQ ID NO: 15), and has the ability to reduce Fe³⁺ to Fe²⁺ and Cu²⁺ to Cu¹⁺ as a metalloreductase (https://www.uniprot.org/uniprot/Q9UHE8; and https://www.ncbi.nlm.nih.gov/protein/NP_036581.1).

### MMP14

MMP14 (matrix metalloproteinase-14) is a protein represented by [NP_004986.1] (SEQ ID NO: 16), and has endopeptidase activity for degrading extracellular matrices and such (https://www.ncbi.nlm.nih.gov/protein/NP_004986.1).

### TNFRSF21

TNFRSF21 (tumor necrosis factor receptor superfamily member 21) is a protein represented by [NP_055267.1] (SEQ ID NO: 17), and is considered to induce apoptosis by activating nuclear factor kappa-B and mitogen-activated protein kinase 8. It is also called Death receptor 6 (DR6) (https://www.ncbi.nlm.nih.gov/protein/NP_055267.1).

### TMPRSS4

TMPRSS4 (transmembrane protease serine 4) is a protein represented by [NP_063947.1] (SEQ ID NO: 18), and it may be a serine protease (https://www.ncbi.nlm.nih.gov/protein/NP_063947.1).

### A. Exemplary Antibodies

In one aspect, the present disclosure provides isolated antibodies that bind to any of the following target proteins: XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4. In certain embodiments, an antibody of the present disclosure binds to an extracellular domain of the above-mentioned target protein. In certain embodiments, an antibody of the present disclosure has cytotoxic activity. In certain embodiments, an antibody of the present disclosure has internalization activity.

In one aspect, the present disclosure provides an antibody that can bind to XPR1 (XPR1-binding antibody).

In certain embodiments, the epitope of the XPR1-binding antibody of the present disclosure is the sequence of any of the following in the XPR1 amino acid sequence represented by SEQ ID NO: 1:

amino acid positions 1 to 108, 111 to 122, 159 to 171, 177 to 216, 423 to 448, 473 to 502, 660 to 670, 674 to 696, 177 to 190, 428 to 448, 660 to 670, 244, 256 to 273, 257 to 270, 258 to 264, 258 to 268, 256 to 270, 258 to 273, 260 to 270, 293 to 314, 336 to 343, 337 to 344, 338 to 341, 340 to 342, 340 to 344, 343 to 344, 340 to 345, 368 to 372, 392 to 398, 397 to 401, 398 to 402, 420 to 442, 420 to 506, 465 to 479, 497 to 507, 498 to 508, 529 to 555, 529 to 570, 582 to 586, 1 to 234, 1 to 236, 293 to 318, 367 to 442, 369 to 473, 500 to 507, 529 to 696, and 589 to 696.

In certain embodiments, the XPR1-binding antibody of the present disclosure is any one of (a1) to (a6):
(a1) an antibody comprising HVR-H1 consisting of the amino acid sequence of SEQ ID NO: 35, HVR-H2 consisting of the amino acid sequence of SEQ ID NO: 36, HVR-H3 consisting of the amino acid sequence of SEQ ID NO: 37, HVR-L1 consisting of the amino acid sequence of SEQ ID NO: 38, HVR-L2 consisting of the amino acid sequence of SEQ ID NO: 39, and HVR-L3 consisting of the amino acid sequence of SEQ ID NO: 40;
(a2) an antibody comprising the VH sequence of SEQ ID NO: 41 and the VL sequence of SEQ ID NO: 42;
(a3) an antibody that binds to the same epitope in XPR1 as the antibody of any one of (a1) to (a2);
(a4) an antibody that competes with the antibody of any one of (a1) to (a2) for binding to XPR1;
(a5) an antibody that blocks the binding of the antibody of any one of (a1) to (a2) to XPR1 by 50% or more in a competitive assay; and
(a6) an antibody comprising a VH sequence having at least 95% sequence identity with the amino acid sequence of SEQ ID NO: 41 and a VL sequence having at least 95% sequence identity with the amino acid sequence of SEQ ID NO: 42.

In one aspect, the present disclosure provides an antibody that can bind to NOX1 (NOX1-binding antibody).

In certain embodiments, the epitope of the NOX1-binding antibody of the present disclosure is the sequence of any of the following in the amino acid sequence represented by SEQ ID NO: 2:

amino acid positions 44 to 54, 131 to 161, 242 to 258, 1 to 4, 1 to 11, 18 to 55, 28 to 44, 31 to 44, 32 to 46, 34 to 50, 70 to 102, 70 to 103, 117 to 176, 120 to 172, 122 to 166, 122 to 172, 124 to 168, 190 to 208, 191 to 204, 223 to 266, 223 to 267, 227 to 269, 228 to 391, 228 to 396, 404, and 420 to 564.

In certain embodiments, the NOX1-binding antibody of the present disclosure is any one of (b1) to (a9):
(b1) an antibody comprising HVR-H1 consisting of the amino acid sequence of SEQ ID NO: 67, HVR-H2 consisting of the amino acid sequence of SEQ ID NO: 68, HVR-H3 consisting of the amino acid sequence of SEQ ID NO: 69, HVR-L1 consisting of the amino acid sequence of SEQ ID NO: 70, HVR-L2 consisting of the amino acid sequence of SEQ ID NO: 71, and HVR-L3 consisting of the amino acid sequence of SEQ ID NO: 72;
(b2) an antibody comprising HVR-H1 consisting of the amino acid sequence of SEQ ID NO: 75, HVR-H2 consisting of the amino acid sequence of SEQ ID NO: 76, HVR-H3 consisting of the amino acid sequence of SEQ ID NO: 77, HVR-L1 consisting of the amino acid sequence of SEQ ID NO: 78, HVR-L2 consisting of the amino acid sequence of SEQ ID NO: 79, and HVR-L3 consisting of the amino acid sequence of SEQ ID NO: 80;
(b3) an antibody comprising the VH sequence of SEQ ID NO: 73 and the VL sequence of SEQ ID NO: 74;
(b4) an antibody comprising the VH sequence of SEQ ID NO: 81 and the VL sequence of SEQ ID NO: 82;
(b5) an antibody that binds to the same epitope in NOX1 as the antibody of any one of (b1) to (b4);
(b6) an antibody that competes with the antibody of any one of (b1) to (b4) for binding to NOX1;
(b7) an antibody that blocks the binding of the antibody of any one of (b1) to (b4) to NOX1 by 50% or more in a competitive assay;
(b8) an antibody comprising a VH sequence having at least 95% sequence identity with the amino acid sequence of SEQ ID NO: 73 and a VL sequence having at least 95% sequence identity with the amino acid sequence of SEQ ID NO: 74; and
(b9) an antibody comprising a VH sequence having at least 95% sequence identity with the amino acid sequence of SEQ ID NO: 81 and a VL sequence having at least 95% sequence identity with the amino acid sequence of SEQ ID NO: 82.

In one aspect, the present disclosure provides an antibody that can bind to MARVELD3 isoform1 (MARVELD3 isoform1-binding antibody).

In certain embodiments, the epitope of the MARVELD3 isoform1-binding antibody of the present disclosure is the sequence of any of the following in the amino acid sequence represented by SEQ ID NO: 3:

amino acid positions 222 to 266, 223 to 269, 227 to 264, 227 to 268, 229 to 263, 231 to 265, 321 to 362, 322 to 357, 323 to 357, 324 to 357, and 324 to 358.

In one aspect, the present disclosure provides an antibody that can bind to MARVELD3 isoform2 (MARVELD3 isoform2-binding antibody).

In certain embodiments, the epitope of the MARVELD3 isoform2-binding antibody of the present disclosure is the sequence of any of the following in the amino acid sequence represented by SEQ ID NO: 4:

amino acid positions 101 to 111, 163 to 198, 1 to 266, 1 to 270, 216 to 271, 222 to 269, 226 to 271, 227 to 268, 227 to 271, 248 to 271, 316 to 364, 322 to 360, 323 to 359, 324 to 360, 326 to 360, and 327 to 360.

In certain embodiments, the MARVELD3 isoform2-binding antibody of the present disclosure is any one of (c1) to (c9):
(c1) an antibody comprising HVR-H1 consisting of the amino acid sequence of SEQ ID NO: 43, HVR-H2 consisting of the amino acid sequence of SEQ ID NO: 44, HVR-H3 consisting of the amino acid sequence of SEQ ID NO: 45, HVR-L1 consisting of the amino acid sequence of SEQ ID NO: 46, HVR-L2 consisting of the amino acid sequence of SEQ ID NO: 47, and HVR-L3 consisting of the amino acid sequence of SEQ ID NO: 48;
(c2) an antibody comprising HVR-H1 consisting of the amino acid sequence of SEQ ID NO: 51, HVR-H2 consisting of the amino acid sequence of SEQ ID NO: 52, HVR-H3 consisting of the amino acid sequence of SEQ ID NO: 53, HVR-L1 consisting of the amino acid sequence of SEQ ID NO: 54, HVR-L2 consisting of the amino acid sequence of SEQ ID NO: 55, and HVR-L3 consisting of the amino acid sequence of SEQ ID NO: 56;
(c3) an antibody comprising the VH sequence of SEQ ID NO: 49 and the VL sequence of SEQ ID NO: 50;
(c4) an antibody comprising the VH sequence of SEQ ID NO: 57 and the VL sequence of SEQ ID NO: 58;
(c5) an antibody that binds to the same epitope in MARVELD3 isoform 1 and/or MARVELD3 isoform 2 as the antibody of any one of (c1) to (c4);
(c6) an antibody that competes with the antibody of any one of (c1) to (c4) for binding to MARVELD3 isoform 1 and/or MARVELD3 isoform 2;
(c7) an antibody that blocks the binding of the antibody of any one of (c1) to (c4) to MARVELD3 isoform 1 and/or MARVELD3 isoform 2 by 50% or more in a competitive assay;
(c8) an antibody comprising a VH sequence having at least 95% sequence identity with the amino acid sequence of SEQ ID NO: 49 and a VL sequence having at least 95% sequence identity with the amino acid sequence of SEQ ID NO: 50; and
(c9) an antibody comprising a VH sequence having at least 95% sequence identity with the amino acid sequence of SEQ ID NO: 57 and a VL sequence having at least 95% sequence identity with the amino acid sequence of SEQ ID NO: 58.

In a further aspect of the present disclosure, an anti-target protein antibody according to any of the above embodiments is a monoclonal antibody, including a chimeric, humanized or human antibody. In one embodiment, an anti-target protein antibody is an antibody fragment, e.g., a Fv, Fab, Fab′, scFv, diabody, or F(ab′)₂ fragment. In another embodiment, the antibody is, for example, a full length IgG antibody or a full length antibody of other antibody class or isotype as defined herein.

In a further aspect, an anti-target protein antibody according to any of the above embodiments may incorporate any of the features, singly or in combination, as described in Sections 1-7 below:

### 1. Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³M, e.g., from 10⁻⁹M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, N.J.) is performed at 25 degrees C. with immobilized antigen CMS chips at −10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab (registered trademark) technology; U.S. Pat. No. 7,041,870 describing K-M MOUSE (registered trademark) technology, and U.S. Patent Application Publication No. US 2007/0061900, describing VelociMouse (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the present disclosure may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for a target protein and the other is for any other antigen. In certain embodiments, bispecific antibodies may bind to two different epitopes of a target protein. Bispecific antibodies may also be used to localize cytotoxic agents to cells which express a target protein. Bispecific antibodies can be prepared as full length antibodies or antibody fragments.

In certain embodiments, a multispecific antibody provided herein is a T cell-redirecting antibody (TRAB). Such antibodies interact with two or more of the target proteins of the present disclosure, that is, proteins that are highly expressed specifically in cancer cells, and a protein expressed in T cells. Thus, they have the effect of enhancing antitumor activities by crosslinking T cells having cytotoxic activity with cancer cells.

Blinatumomab, which is a BiTE molecule, and Catumaxomab are known as bispecific antibodies that recognize a protein expressed on T cells (CD3c or TCR) and a protein expressed on cancer cells (a cancer antigen). These molecules can bind to a cancer antigen and the CD3c chain expressed on a T cell with each of their two antigen-binding domains (scFv or Fab), and form intercellular crosslinks between the T cells and the cancer antigen-expressing cells. This way, such T cell-redirecting antibodies can use T cells as effector cells to induce strong cytotoxic activity against cancer antigen-expressing cells.

In certain embodiments, a multispecific antibody of the present disclosure comprises a binding domain for a target protein of the present disclosure and a T cell receptor complex-binding domain and optionally has T cell dependent cytotoxic activity. Such a multispecific antibody of the present disclosure can redirect T cells to cells that express the target protein of the present disclosure on the cell surface, thereby inducing T cell-mediated cytotoxic activity against the cells. In certain embodiments, a multispecific antibody of the present disclosure comprises an Fc region that has reduced Fcγ receptor-binding activity, and optionally comprises an Fc region that has a lower Fcγ receptor-binding activity than the Fc region of IgG1, IgG2, IgG3, or IgG4. In certain embodiments, a multispecific antibody of the present disclosure comprises one binding domain for a target protein of the present disclosure. The T cell receptor complex-binding domain contained in the multispecific antibody of the present disclosure is, in one embodiment, a T cell receptor-binding domain having activity to bind to a T cell receptor, or in another embodiment, a CD3-binding domain having CD3-binding activity. In certain embodiments, the CD3 binding domain contained in the multispecific antibody of the present disclosure is a domain that is capable of binding to the CD3c chain and optionally comprises an antibody heavy chain variable region and an antibody light chain variable region.

In certain embodiments, the CD3-binding domain contained in a multispecific antibody of the present disclosure is any one of the following:
(d1) a domain comprising an antibody variable region having HVR-H1 consisting of the amino acid sequence of SEQ ID NO: 59, HVR-H2 consisting of the amino acid sequence of SEQ ID NO: 60, HVR-H3 consisting of the amino acid sequence of SEQ ID NO: 61, HVR-L1 consisting of the amino acid sequence of SEQ ID NO: 62, HVR-L2 consisting of the amino acid sequence of SEQ ID NO: 63, and HVR-L3 consisting of the amino acid sequence of SEQ ID NO: 64;
(d2) a domain comprising an antibody variable region having the VH sequence of SEQ ID NO: 65 and the VL sequence of SEQ ID NO: 66;
(d3) a domain comprising an antibody variable region that binds to the same epitope in CD3 as the antibody variable region of any one of (d1) to (d2);
(d4) a domain comprising an antibody variable region that competes with the antibody variable region of any one of (d1) to (d2) for binding to CD3;
(d5) a domain comprising an antibody variable region that blocks the binding of the antibody variable region of any one of (d1) to (d2) to CD3 by 50% or more in a competitive assay; and
(d6) a domain comprising an antibody variable region that has a VH sequence having at least 95% sequence identity with the amino acid sequence of SEQ ID NO: 65 and a VL sequence having at least 95% sequence identity with the amino acid sequence of SEQ ID NO: 66.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting Fab” or “DAF” comprising an antigen binding site that binds to a target protein as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e g, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc region variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region represented by SEQ ID NO: 6, 19, 20, or 21, respectively) comprising an amino acid modification (e.g. a substitution) at one or more amino acid positions.

In certain embodiments, the present disclosure contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, Calif.; and CytoTox 96 (registered trademark) non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Antibody Production Methods

Methods for producing antibodies having the desired binding activity are known to those skilled in the art, and the antibodies may be obtained as polyclonal or monoclonal antibodies. Monoclonal antibodies derived from mammals may be suitably produced as the antibodies of the present disclosure. Such mammalian-derived monoclonal antibodies include antibodies produced by hybridomas and antibodies produced by host cells transformed with an expression vector carrying an antibody gene by genetic engineering techniques.

There is no particular limitation on the mammal to be immunized for obtaining antibodies. It is preferable to select the mammal by considering its compatibility with the parent cells to be used in cell fusion for hybridoma production. In general, rabbits, monkeys, and rodents such as mice, rats, and hamsters are suitably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with Phosphate-Buffered Saline (PBS), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein; and
there is no need to purify the antigen for immunization.

In order to prepare a monoclonal antibody of the present disclosure using DNA immunization, first, a DNA expressing an antigen protein is administered to an animal to be immunized. The antigen protein-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized.

After immunizing a mammal as described above, an increase in the titer of an antigen-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immune cells. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:

P3(P3x63Ag8.653) (J. Immunol. (1979) 123 (4), 1548-1550);

P3x63Ag8U.1 (Current Topics in Microbiology and Immunology (1978)81, 1-7);

NS-1 (C. Eur. J. Immunol. (1976)6 (7), 511-519);

MPC-11 (Cell (1976) 8 (3), 405-415);

SP2/0 (Nature (1978) 276 (5685), 269-270);

FO (J. Immunol. Methods (1980) 35 (1-2), 1-21);

S194/5.XX0.BU.1 (J. Exp. Med. (1978) 148 (1), 313-323);

R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be arbitrarily set, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37° C. is added thereto at a concentration of generally 30% to 60% (w/v). The mixed solution is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Culture is continued in the above medium using the HAT medium for a period of time sufficient to kill cells other than the desired hybridomas (non-fused cells). Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Culture is continued in the above medium using the HAT medium for a period of time sufficient to kill cells other than the desired hybridomas (non-fused cells). Specifically, desired hybridomas can be selected by culture for generally several days to several weeks. Then, hybridomas producing the desired antibody are screened and singly cloned by conventional limiting dilution methods.

Screening and single cloning of desired antibodies can be suitably performed by screening methods based on known antigen-antibody reaction. For example, a desired antibody can be selected by screening using fluorescence activated cell sorting (FACS). FACS is a system that enables measurement of the binding of an antibody to cell surface by analyzing cells contacted with a fluorescent antibody using laser beam, and measuring the fluorescence emitted from individual cells.

To screen for hybridomas that produce a monoclonal antibody of the present disclosure by FACS, cells that express the antigen bound by the produced antibody are first prepared. Preferred cells used for screening are mammalian cells that are forced to express the antigen. By using mammalian cells that are used as the host cell but have not been transformed as a control, the activity of an antibody to bind to the cell-surface antigen can be selectively detected. Specifically, hybridomas producing a desired monoclonal antibody can be obtained by selecting hybridomas that produce an antibody which binds to cells forced to express the antigen but not to the host cell.

Alternatively, cells expressing the antigen of interest are immobilized and the activity of an antibody to bind to the antigen-expressing cells can be assessed based on the principle of ELISA. For example, antigen-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium. The hybridomas can be stored in liquid nitrogen for a long period.

The above hybridomas are cultured by a conventional method, and desired monoclonal antibodies can be obtained from the culture supernatants. Alternatively, the hybridomas are administered to and grown in compatible mammals, and monoclonal antibodies can be obtained from the ascites. The former method is suitable for obtaining antibodies with high purity.

Antibodies that are encoded by antibody genes cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Generally, to obtain a cDNA encoding the antibody variable region (V region), total RNA is first extracted from hybridomas. For example, the following methods can be used as methods for extracting mRNAs from cells:
the guanidine ultracentrifugation method (Biochemistry (1979) 18(24), 5294-5299), and
the AGPC method (Anal. Biochem. (1987) 162(1), 156-159).

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Corporation) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding γ1, γ2a, γ2b, and γ3 heavy chains and κ and λ light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

Immunoglobulins composed of a combination of heavy and light chains may be reshaped using the thus amplified PCR products. A desired antibody can be selected by screening using the antigen-binding activity of a reshaped immunoglobulin as an indicator. The screening can be carried out, for example, by the following steps:

(1) contacting a desired antigen-expressing cell with an antibody comprising the V region encoded by a cDNA obtained from a hybridoma;

(2) detecting the binding of the antibody to the antigen-expressing cell; and

(3) selecting an antibody that binds to the antigen-expressing cell.

Methods for detecting the binding of an antibody to the antigen-expressing cells are known. Specifically, the binding of an antibody to the antigen-expressing cells can be detected by the above-described techniques such as FACS. Fixed samples of the antigen-expressing cells may be appropriately used to assess the binding activity of an antibody.

For antibody screening methods that use the binding activity as an indicator, panning methods that use phage vectors can also be used suitably. Screening methods using phage vectors are advantageous when the antibody genes are obtained from a polyclonal antibody-expressing cell population as heavy-chain and light-chain subclass libraries. Genes encoding the heavy-chain and light-chain variable regions can be linked by an appropriate linker sequence to form a single-chain Fv (scFv). Phages expressing scFv on their surface can be produced by inserting a scFv-encoding gene into a phage vector. The phages are contacted with an antigen of interest. Then, a DNA encoding scFv having the binding activity of interest can be isolated by collecting phages bound to the antigen. This process can be repeated as necessary to enrich scFv having the binding activity of interest.

After isolation of the cDNA encoding the V region of the antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, a “chimeric antibody” means that the origin of the constant region is different from that of the variable region. Thus, in addition to mouse/human heterochimeric antibodies, human/human allochimeric antibodies are included in the chimeric antibodies of the present disclosure. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA that encodes a desired antibody constant region (C region). A chimeric antibody expression vector is constructed by fusing in-frame two genes digested with the same combination of restriction enzymes.

To produce a monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. The signal sequence is cleaved from the carboxyl terminus of the expressed polypeptide, and the resulting antibody can be secreted to the outside of cells. Then, appropriate host cells are transformed with the expression vector, and recombinant cells expressing the antibody-encoding DNA can be obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors inserted with the H chain and L chain. Alternatively, host cells can be transformed with a single expression vector into which DNAs encoding the H and L chains are inserted (see WO 94/11523).

There are many known combinations of host cells and expression vectors for antibody preparation by introducing isolated antibody genes into appropriate hosts. All these expression systems are applicable to isolation of the domains that bind to the target proteins of the present disclosure described above and T cell receptor complex-binding domain.

Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells:

yeasts: the Saccharomyces genus such as Saccharomyces cerevisiae, and the Pichia genus such as Pichia pastoris; and
filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat (3-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Bio/Technology (1994) 12 (7), 699-702).

When an antigen-binding molecule described herein is administered to human, an antigen-binding domain derived from a genetically recombinant antibody that has been artificially modified to reduce the heterologous antigenicity against human and such, can be appropriately used as the various binding domains in the molecule when domains comprising an antibody variable region are used. Such genetically recombinant antibodies include, for example, humanized antibodies. These modified antibodies are appropriately produced by known methods.

An antibody variable region used to produce the various binding domains of antigen-binding molecules described herein is generally formed by three complementarity-determining regions (CDRs) that are separated by four framework regions (FRs). CDR is a region that substantially determines the binding specificity of an antibody. The amino acid sequences of CDRs are highly diverse. On the other hand, the FR-forming amino acid sequences often have high identity even among antibodies with different binding specificities. Therefore, generally, the binding specificity of a certain antibody can be introduced into another antibody by CDR grafting.

A humanized antibody is also called a reshaped human antibody. Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the PR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length V region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856).

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

In addition to the phage display method, techniques that use a cell-free translation system, techniques for displaying antigen-binding molecules on the surface of viruses or cells, and techniques that use emulsions are also known as techniques for obtaining human antibodies by panning using human antibody libraries. For example, the ribosome display method where a complex is formed between the translated protein and mRNA via the ribosome by removing the stop codon and such, the cDNA display method or the mRNA display method where a genetic sequence and the translated protein are covalently linked using a compound such as puromycin, the CIS display method where a complex is formed between the gene and the translated protein using a nucleic acid-binding protein, or such may be used as techniques of using a cell-free translation system. For the technique of presenting antigen-binding molecules on the surface of cells or viruses, besides the phage display method, the E. coli display method, Gram-positive bacteria display method, yeast display method, mammalian cell display method, virus display method, and such may be used. As a technique that uses emulsions, the in vitro virus display method which involves incorporating genes and translation-related molecules into an emulsion, and such may be used. These methods are already publicly known (Nat Biotechnol. 2000 December; 18(12):1287-92; Nucleic Acids Res. 2006; 34(19): e127; Proc Natl Acad Sci USA. 2004 Mar. 2; 101(9):2806-10; Proc Natl Acad Sci USA. 2004 Jun. 22; 101(25):9193-8; Protein Eng Des Sel. 2008 April; 21(4):247-55; Proc Natl Acad Sci USA. 2000 Sep. 26; 97(20):10701-5; MAbs. 2010 September-October; 2(5):508-18; and Methods Mol Biol. 2012, 911:183-98).

### C. Assays

Anti-target protein antibodies provided herein may be identified, screened for, or characterized for their physical/chemical properties and/or biological activities by various assays known in the art.

### 1. Binding Assays and Other Assays

In one aspect, an antibody of the present disclosure is tested for its antigen binding activity, e.g., by known methods such as ELISA, Western blot, etc.

### 2. Activity Assays

In one aspect, assays are provided for identifying antibodies of the present disclosure thereof having biological activity. Biological activity may include, e.g., cytotoxic activity (ADCC activity, CDC activity, and such) and internalization activity. Antibodies having such biological activity in vivo and/or in vitro are also provided.

### D. Immunoconjugates

The present disclosure also provides immunoconjugates comprising an antibody of the present disclosure conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu. When the radioconjugate is used for detection, it may comprise a radioactive atom for scintigraphic studies, for example Tc-99m or ¹²³I, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### E. Methods and Compositions for Diagnostics and Detection

In certain embodiments, any of the anti-target protein antibodies provided herein is useful for detecting the presence of a target protein in a biological sample. The term “detecting” as used herein encompasses quantitative or qualitative detection. In certain embodiments, a biological sample comprises a cell or tissue, such as a cell or tissue of heart, liver, lung, kidney, spleen, large intestine, or bone marrow.

In one embodiment, an antibody of the present disclosure for use in a method of diagnosis or detection is provided. In a further aspect, a method of detecting the presence of a target protein in a biological sample is provided. In certain embodiments, the method comprises contacting the biological sample with an antibody as described herein under conditions permissive for binding of the antibody as described herein to the target protein, and detecting whether a complex is formed between the antibody and the target protein. Such method may be an in vitro or in vivo method. In one embodiment, an antibody of the present disclosure is used to select subjects eligible for therapy with an antibody of the present disclosure, e.g. where the target protein is a biomarker for selection of patients.

Exemplary disorders that may be diagnosed using an antibody of the present disclosure include cancers such as lung cancer (e.g. lung adenocarcinoma) and colorectal cancer.

In certain embodiments, labeled anti-target protein antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

In one aspect, the present disclosure provides pharmaceutical formulations of an anti-target protein antibody as described herein. In certain embodiments, a pharmaceutical formulation of the present disclosure is an agent for treating, preventing, or diagnosing cancer. Such formulations are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated or prevented, preferably those with complementary activities that do not adversely affect each other. For example, it may be desirable to further provide a cytotoxic agent. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the antibodies provided herein may be used in therapeutic methods. In one aspect, an antibody of the present disclosure for use as a medicament is provided. In further aspects, an antibody of the present disclosure for use in treating cancer is provided. In certain embodiments, an antibody of the present disclosure for use in a method of treatment is provided. In certain embodiments, the present disclosure provides an antibody of the present disclosure for use in a method of treating an individual having cancer (e.g. lung cancer or colorectal cancer) comprising administering to the individual an effective amount of the antibody of the present disclosure. In one such embodiment, the method further comprises administering to the individual an effective amount of at least one additional therapeutic agent, e.g., as described below. In further embodiments, the present disclosure provides an antibody of the present disclosure for use in inducing cytotoxic activity. In certain embodiments, the present disclosure provides an antibody of the present disclosure for use in a method of inducing cytotoxic activity in an individual comprising administering to the individual an effective of the antibody of the present disclosure to induce cytotoxic activity. An “individual” according to any of the above embodiments is preferably a human.

In a further aspect, the present disclosure provides the use of an antibody of the present disclosure in the manufacture or preparation of a medicament. In one embodiment, the medicament is for treatment of cancer (e.g. lung cancer or colorectal cancer). In a further embodiment, the medicament is for use in a method of treating cancer (e.g. lung cancer or colorectal cancer) comprising administering to an individual having cancer (e.g. lung cancer or colorectal cancer) an effective amount of the medicament. In one such embodiment, the method further comprises administering to the individual an effective amount of at least one additional therapeutic agent, e.g., as described below. In a further embodiment, the medicament is for inducing cytotoxic activity. In a further embodiment, the medicament is for use in a method of inducing cytotoxic activity in an individual comprising administering to the individual an amount effective of the medicament to induce cytotoxic activity. An “individual” according to any of the above embodiments may be a human.

In one embodiment of the above-mentioned aspect, in one embodiment of the aspect, the present disclosure provides an antibody or a pharmaceutical of the present disclosure for use in treatment of lung cancer, wherein the antibody or pharmaceutical is for administration to a lung cancer patient who has been selected by the following: assessing the presence or absence of one or more selected from an EGFR driver mutation, a BRAF driver mutation, an ERBB2 driver mutation, ALK fusion gene expression, and RET/ROS1 fusion gene expression in a biological sample obtained from a lung cancer patient, and selecting a lung cancer patient who does not have one or more selected from an EGFR driver mutation, a BRAF driver mutation, an ERBB2 driver mutation, ALK fusion gene expression, and RET/ROS1 fusion gene expression, as a responder to treatment with the antibody or pharmaceutical of the present disclosure. In another embodiment, the present disclosure provides an antibody or a pharmaceutical of the present disclosure for use in treatment of colorectal cancer, wherein the antibody or pharmaceutical is for administration to a colorectal cancer patient who has been selected by the following: assessing the presence or absence of a KRAS mutation in a biological sample obtained from a colorectal cancer patient, and selecting a colorectal cancer patient with a KRAS mutation as a responder to treatment with the antibody or pharmaceutical of the present disclosure. Methods for assessing the presence or absence of EGFR driver mutations, BRAF driver mutations, ERBB2 driver mutations, ALK fusion gene expression, RET/ROS1 fusion gene expression, and KRAS mutations are known in the art, and examples include the methods described in the Examples herein.

In a further aspect, the present disclosure provides a method for treating cancer (e.g. lung cancer and colorectal cancer) (synonymous with a method for treating an individual having cancer). In one embodiment, the method comprises administering to an individual having such cancer (e.g. lung cancer or colorectal cancer) an effective amount of an antibody of the present disclosure. In one such embodiment, the method further comprises administering to the individual an effective amount of at least one additional therapeutic agent, as described below. An “individual” according to any of the above embodiments may be a human.

In an embodiment of the above-mentioned aspect, the above method comprises assessing the presence or absence of one or more selected from an EGFR driver mutation, a BRAF driver mutation, an ERBB2 driver mutation, ALK fusion gene expression, and RET/ROS1 fusion gene expression in a biological sample obtained from an individual who has lung cancer; selecting an individual who does not have one or more selected from an EGFR driver mutation, a BRAF driver mutation, an ERBB2 driver mutation, ALK fusion gene expression, and RET/ROS1 fusion gene expression as a responder to treatment with the antibody of the present disclosure; and administering the antibody of the present disclosure to the selected individual. In another embodiment, the above method comprises assessing the presence or absence of a KRAS mutation in a biological sample obtained from an individual who has colorectal cancer; selecting an individual with a KRAS mutation as a responder to treatment with the antibody of the present disclosure; and administering the antibody of the present disclosure to the selected individual.

In a further aspect, the present disclosure provides a method for inducing cytotoxic activity in an individual. In one embodiment, the method comprises administering to the individual an effective amount of an antibody of the present disclosure to include cytotoxic activity. In one embodiment, an “individual” is a human.

In a further aspect, the present disclosure provides pharmaceutical formulations comprising any of the antibodies provided herein, e.g., for use in any of the above therapeutic methods. In one embodiment, a pharmaceutical formulation comprises any of the antibodies provided herein and a pharmaceutically acceptable carrier. In another embodiment, a pharmaceutical formulation comprises any of the antibodies provided herein and at least one additional therapeutic agent, e.g., as described below.

Antibodies of the present disclosure can be used either alone or in combination with other agents in a therapy. For instance, an antibody of the present disclosure may be co-administered with at least one additional therapeutic agent. In certain embodiments, an additional therapeutic agent is a cytotoxic agent.

Such combination therapies noted above encompass combined administration (where two or more therapeutic agents are included in the same or separate formulations), and separate administration, in which case, administration of the antibody of the present disclosure can occur prior to, simultaneously, and/or following, administration of the additional therapeutic agent or agents. In one embodiment, administration of the antibody of the present disclosure and administration of an additional therapeutic agent occur within about one month, or within about one, two or three weeks, or within about one, two, three, four, five, or six days, of each other. Antibodies of the present disclosure can also be used in combination with radiation therapy.

An antibody of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the present disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the present disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate of the present disclosure in place of or in addition to an antibody of the present disclosure.

### H. Articles of Manufacture

In another aspect of the present disclosure, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the present disclosure. The label or package insert indicates that the composition is used for treating the condition of choice. Moreover, the article of manufacture may comprise (a) a first container with a composition contained therein, wherein the composition comprises an antibody of the present disclosure; and (b) a second container with a composition contained therein, wherein the composition comprises a further cytotoxic or otherwise therapeutic agent. The article of manufacture in this embodiment of the present disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the present disclosure in place of or in addition to an antibody of the present disclosure.

### EXAMPLES

### (Example 1) Human Tissues and Gene Analyses Thereof

### (1-1) Obtaining Colorectal Cancer Tissues, Lung Cancer Tissues, Adjacent Normal Tissues, and Normal Tissues

Human tissues to be analyzed were purchased from Asterand, ILS Bio, and All Cells. For colorectal cancer tissues and normal tissues adjacent to colorectal cancer tissue, 12 pairs of a colorectal cancer tissue and a normal tissue adjacent to the colorectal cancer tissue derived from the same donors (stage III or IV) (from 12 donors, a total of 24 specimens) were obtained. Additionally, two specimens of cancer tissue that had metastasized to the lymph node were obtained from each of two of the 12 donors. In total, 28 specimens were obtained.

For lung cancer tissues and normal tissues adjacent to lung cancer tissue, 14 specimens of lung adenocarcinoma tissues and 10 specimens of normal tissues adjacent to lung adenocarcinoma were obtained. Of these two sets of a cancer tissue and an adjacent normal tissue were derived from the same donors, and the others were not.

For normal tissues, three specimens of normal or adjacent normal tissues each from the heart, liver, lung, kidney, spleen, colon, and bone marrow were obtained.

### (1-2) Analysis of ALK, RET Fusion Gene in Human Lung Cancer Tissues (RNA-Seq Analysis)

RNA-seq analysis (Takara Bio) was performed to examine whether the lung cancer tissues obtained in Example 1-1 had a driver fusion gene (ALK, RET/ROS1). Total RNA was extracted from two to three tissue pieces of approximately 3 mm in size using NucleoSpinTissue (Macherey-Nagel) by the method recommended by the manufacturer. A DNA library was prepared from the total RNA sample using TruSeq RNA Sample Prep Kit v2 (Illumina) by the method recommended by the manufacturer. The prepared DNA libraries were mixed, and single-lane, 100-base paired-end sequencing was performed using Illumina sequencer HiSeq, and nucleotide sequences (read sequences) were obtained by the software supplied with the sequencer. Fusion gene analyses were performed using STAR-Fusion (https://github.com/STAR-Fusion/STAR-Fusion).

The results revealed that among the 14 lung cancer tissue samples obtained, none had a RET/ROS1 fusion gene, and one had an ALK fusion gene (485475RF) (Table 2).

### (1-3) Driver Mutation Analysis of Human Colorectal Cancer Tissue and Lung Cancer Tissue (Exome-Seq Analysis)

Exome-seq analysis (Takara Bio) was performed to examine whether the cancer tissues obtained in Example 1-1 had driver mutations (KRAS, EGFR, BRAF, ERBB2). Genomic DNA was extracted from two to three tissue pieces of approximately 3 mm in size using NucleoSpinTissue (Macherey-Nagel) by the method recommended by the manufacturer. A DNA library was prepared from the genomic DNA sample using SureSelect XT Human All Exon V6 (Agilent) by the method recommended by the manufacturer. The prepared DNA libraries were mixed, and 100-base paired-end analysis was performed using Illumina sequencer HiSeq, and nucleotide sequences (read sequences) were obtained by the software supplied with the sequencer. Mutation analysis was performed using CLC Genomics Server (QIAGEN).

The results showed that, for the colorectal cancer tissues, 7 out of the 12 donor-derived samples (1170614F, 1171659F, 39694FT, 39697FT, 39698FT, 39705FT, and 40644FT) were found to have a KRAS driver mutation. For the lung cancer tissues, three samples (1191924F, 1160304F, and 1245371F) were found to have an EGFR driver mutation, two samples (1186142F and 1209913F) a BRAF driver mutation, and three samples (1177695F, 1185682F, and 463102XF) a KRAS driver mutation (Table 2).

TABLE 2
Summary of results of driver-mutation and fusion-gene
analyses on colorectal cancer and lung cancer tissues.

| | | | | | | |
|---|---|---|---|---|---|---|
| | | | | | | Fusion |
| ID | Tissue | KRAS | GFR | BRAF | ERBB2 | gene |

| | | | | | |
|---|---|---|---|---|---|
| 1119061F | colorectal | | | | |
| | cancer | | | | |
| 1136277F | colorectal | | | | |
| | cancer | | | | |
| 1145808F | colorectal | | | | |
| | cancer | | | | |
| 1170614F | colorectal | Gly12Asp | | | |
| | cancer | | | | |
| 1171659F | colorectal | Gly12Val | | | |
| | cancer | | | | |
| ILS39694FT1 | colorectal | Gly12Cys | | | |
| | cancer | | | | |
| ILS39697FT1 | colorectal | Gln61Arg | | | |
| | cancer | | | | |
| ILS39698FT1 | colorectal | Gly13Asp | | | |
| | cancer | | | | |
| ILS39705FT1 | colorectal | Gly12Ser | | | |
| | cancer | | | | |
| ILS40183FT4 | colorectal | | | | |
| | cancer | | | | |
| ILS40636FT4 | colorectal | | | | |
| | cancer | | | | |
| ILS40636FM1 | colorectal | | | | |
| | cancer, | | | | |
| | metastasis | | | | |
| | to lymph | | | | |
| | node | | | | |
| ILS40636FM2 | colorectal | | | | |
| | cancer, | | | | |
| | metastasis | | | | |
| | to lymph | | | | |
| | node | | | | |
| ILS40644FT1 | colorectal | Gly12Val | | | |
| | cancer | | | | |
| ILS40644FM1 | colorectal | Gly12Val | | | |
| | cancer, | | | | |
| | metastasis | | | | |
| | to lymph | | | | |
| | node | | | | |
| ILS40644FM2 | colorectal | Gly12Val | | | |
| | cancer, | | | | |
| | metastasis | | | | |
| | to lymph | | | | |
| | node | | | | |
| 463118ZF | lung cancer | | | | |
| 1081042F | lung cancer | | | | |
| 1177695F | lung cancer | Gly12Cys | | | |
| 1184871F | lung cancer | | | | |
| 1185682F | lung cancer | Gly12Cys | | | |
| 1204668F | lung cancer | | | | |
| 1245371F | lung cancer | | Gly719Ala | | |
| 463102XF | lung cancer | Gly12Cys | | | |
| ILS31537 D3 | lung cancer | | | | |
| 485475RF | lung cancer | | | | ALK |
| 1186142F | lung cancer | | | Gly466Ala | |
| 1191924F | lung cancer | | Glu746_Ala750del | | |
| 1160304F | lung cancer | | Leu858Arg | | |
| 1209913F | lung cancer | | | Val600Glu | |

The detected driver mutations are summarized in Table 2. ILS40636FM1, ILS40636FM2, ILS40644FM1, and ILS40644FM2 are results from cancer tissues having metastasized to the lymph node, and the others are results from colorectal cancer and lung cancer tissues.

### (Example 2) Protein Expression Analysis of Human Tissues (Proteomics Analysis)

The tissue block obtained in Example 1-1 in a frozen state was placed in a mortar containing liquid nitrogen, and broken into tissue pieces of about 3 mm in size by hitting with a pestle. Two to three tissue pieces were transferred to a tube, and finely ground using a multi-bead shocker (Yasui Kikai). A tissue lysis buffer (2% deoxycholic acid/100 mM ammonium bicarbonate, Protease inhibitor cocktail (Roche), and PhosSTOP (Roche)) was added to the ground tissue, and the tissue was ultrasonically disrupted using an acoustic solubilizer (Covaris) or Bioruptor (Sonicbio), and centrifuged at 15,000 rpm for 30 minutes to remove insoluble materials. Then, the supernatant was collected as a tissue extract. For the bone marrow, the tissue was washed with PBS, and the tissue lysis buffer was added to precipitated cells. Then, a tissue extract was obtained in the same manner as the other tissues.

Proteins in the tissue extract were reduced with dithiothreitol, alkylated with iodoacetamide, and then precipitated by TCA/acetone precipitation. The proteins were dissolved in 8 M urea/400 mM ammonium bicarbonate solution, and the protein solution was diluted four times with distilled water. The diluted protein solution was subjected to protein digestion with lysyl endopeptidase and trypsin using Rapid Enzyme Digestion System (REDS) (AMR) to obtain a peptide solution. The peptide solution was desalted using Monospin C18 (GL Science) by the method recommended by the manufacturer. Thereafter, the desalted peptide solution was fractionated using Pierce™ High pH Reversed-Phase Peptide Fractionation Kit (ThermoFisher Scientific) or AssayMAP Bravo cartridge RP-S (Agilent) by the method recommended by the manufacturer, and each fraction was analyzed by an LC-MS analysis system in which Q-Exactive (ThermoFisher Scientific) was coupled to a nano-LC system (Ultimate3000, Dionex).

Raw data after the LC-MS analyses were used for database search using MaxQuant (http://www.biochem.mpg.de/5111795/maxquant) to identify proteins and obtain quantification values (iBAQ). The database search was performed with the following parameters:

Taxonomy: uniprot human, Fixed modification: carbamidomethylation (C), and Variable modification: oxidation (M); deamidation (NQ), Acetyl (protein N-term), and FDR (protein, peptide)<1%.
The quantification values were calculated by performing the following processing on the MaxQuant output file “proteinGroups.txt”:
1. Sum up the signal intensities of protein groups assigned to the same gene to calculate the intensity at the gene level.
2. For each gene, normalize the intensity with the number of fragments that can be generated by trypsinization to calculate the iBAQ score.
3. For each sample, perform normalization such that the score average is 1,000,000.

A total of more than 10,000 proteins were identified, and an iBAQ value was obtained for each protein. Cell surface proteins that showed high protein expression in cancer and low expression in adjacent normal tissues and normal tissues—7 proteins from colorectal cancer (SPINT2, MARVELD3, MANSC1, NOX1, SLC12A2, CDCP1, and CEACAM5) and 10 proteins from lung cancer (SPINT2, PVRL4, SEZ6L2, XPR1, FLVCR1, SLC7A5, STEAP1, MMP14, TNFRSF21, and TMPRSS4)—were selected as target antigen candidates

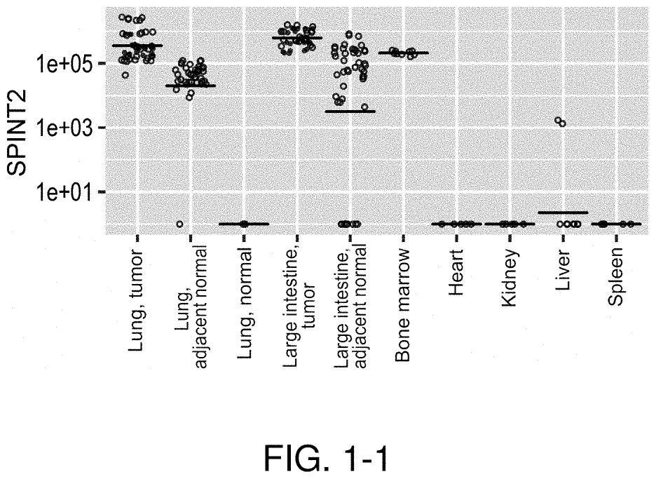

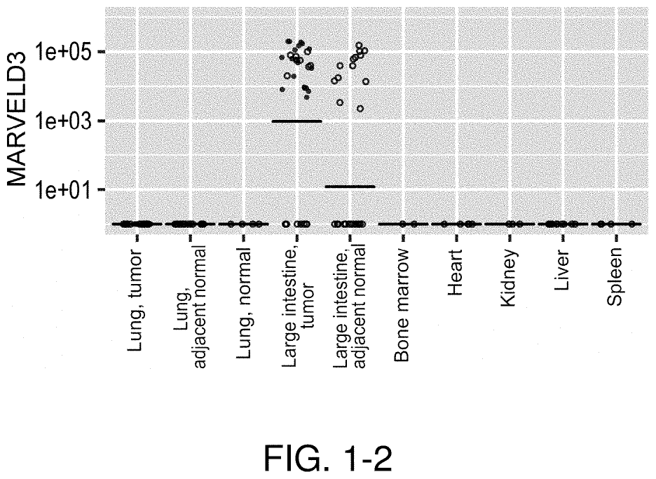

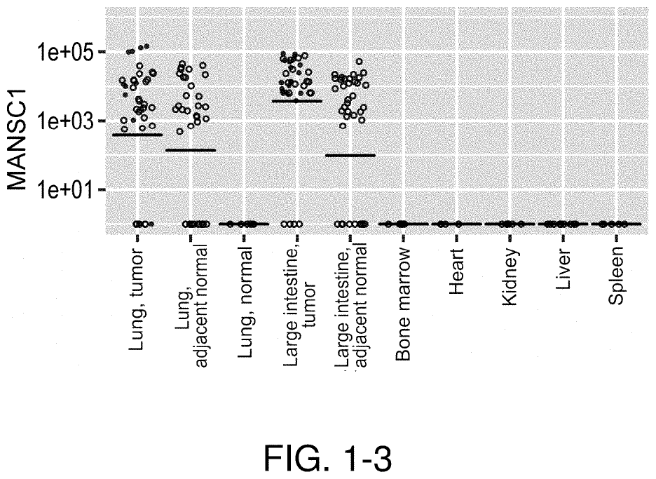

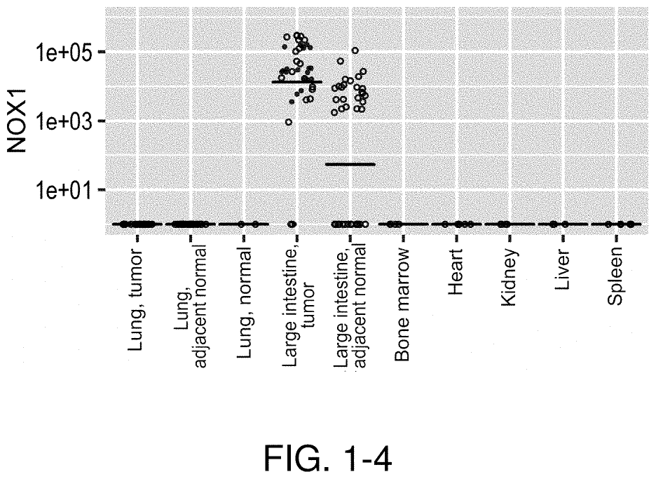

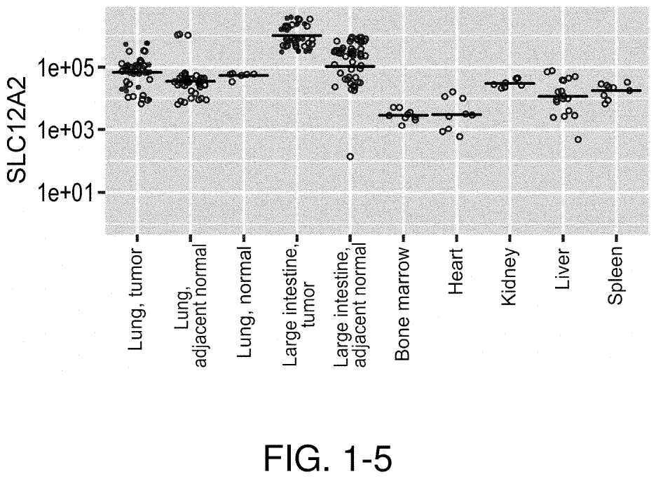

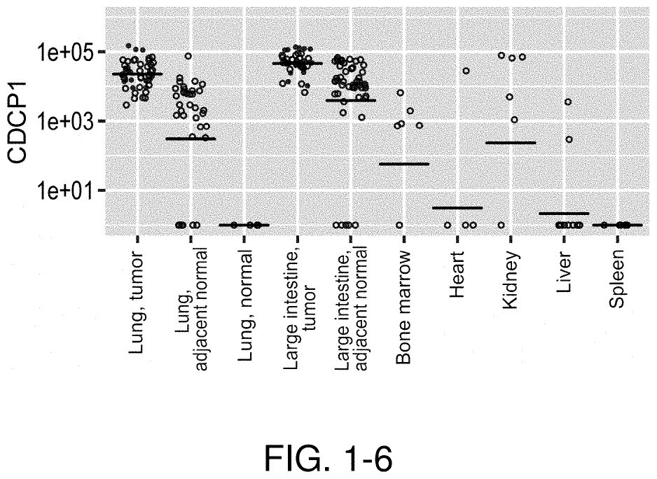

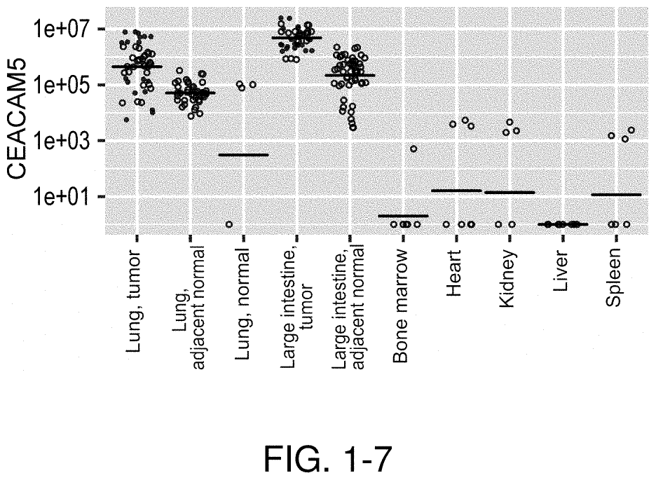

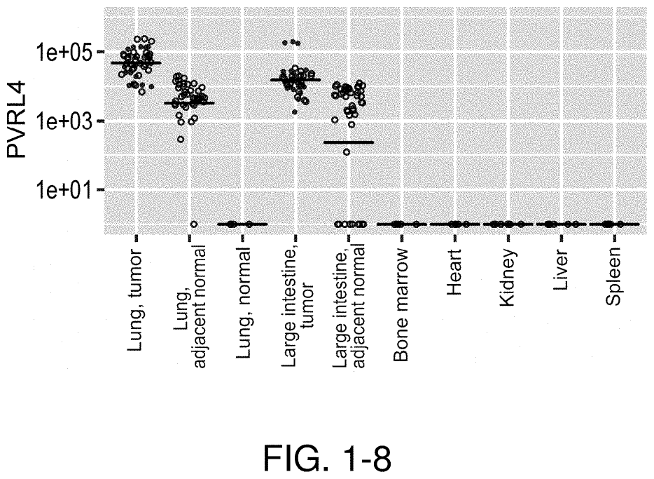

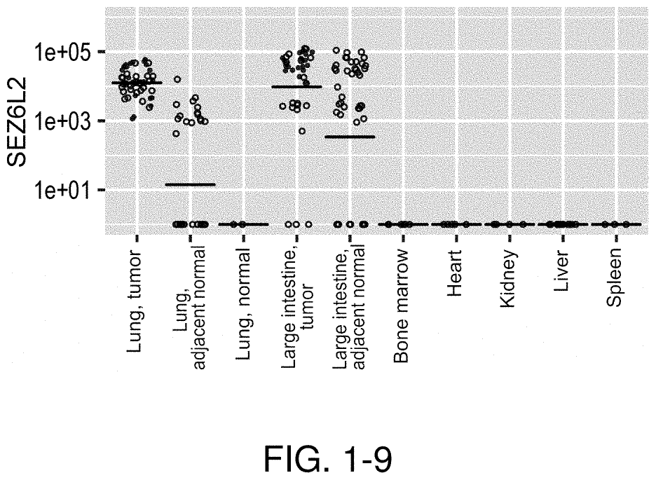

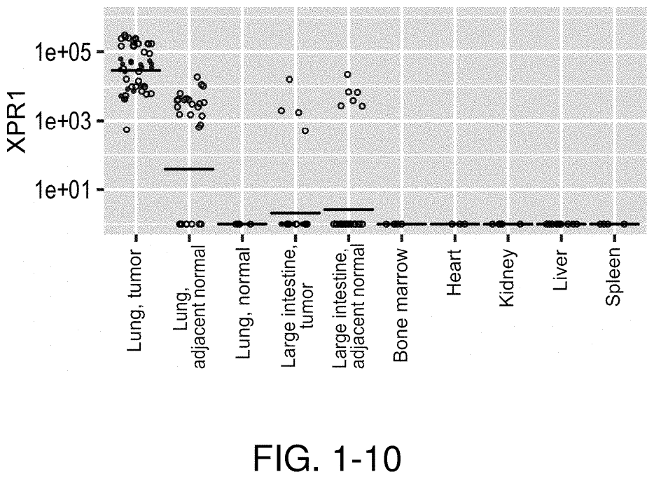

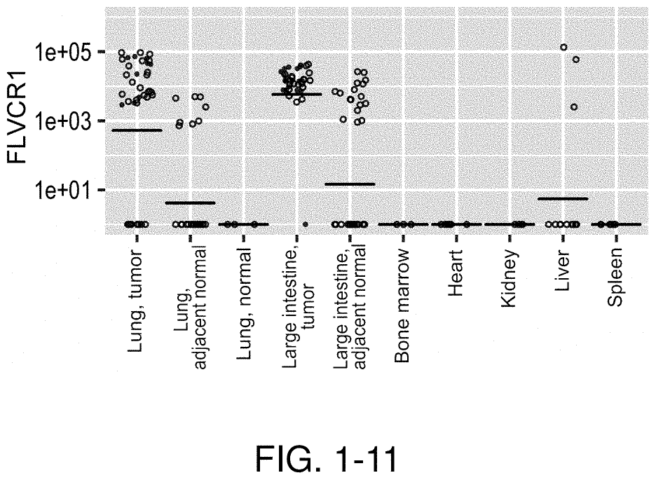

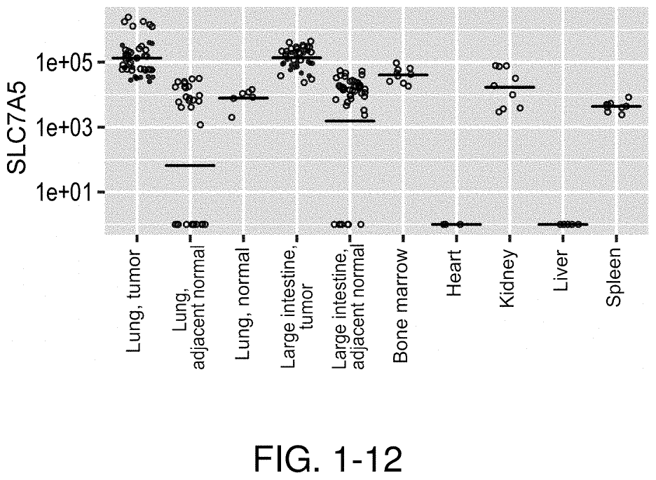

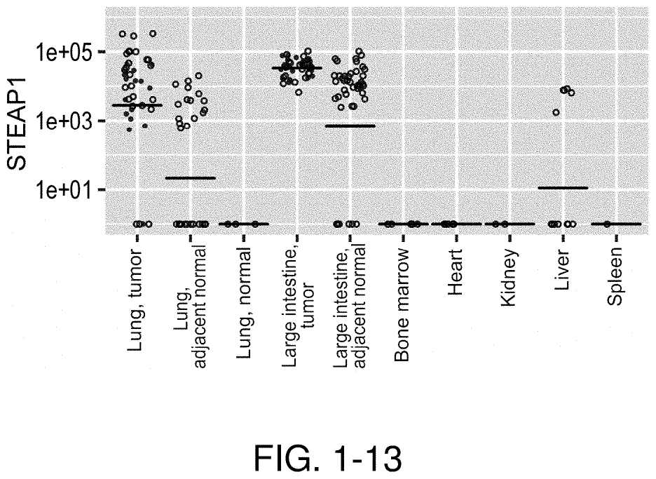

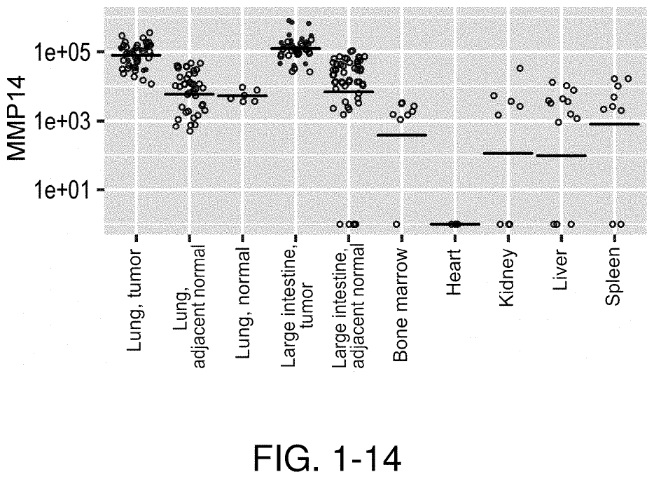

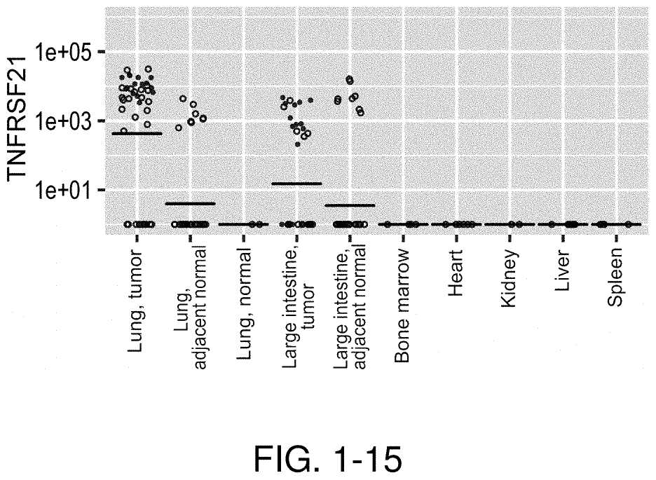

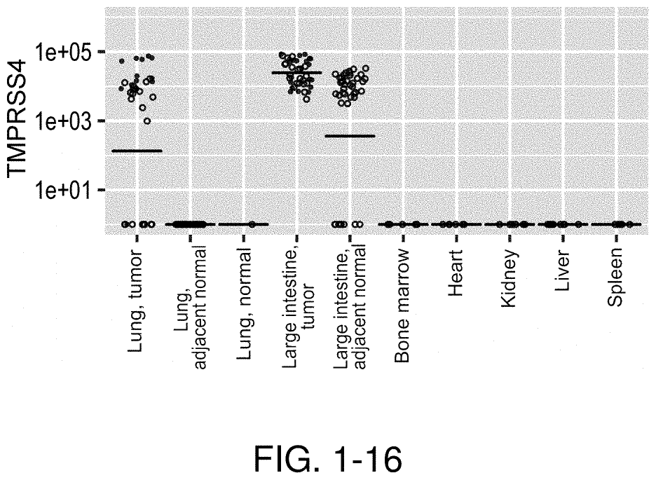

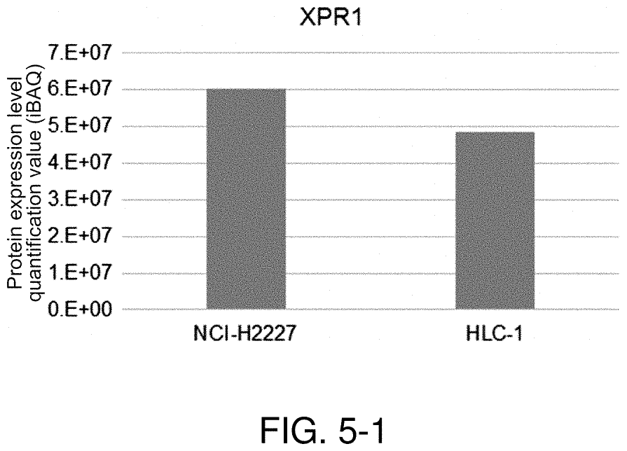

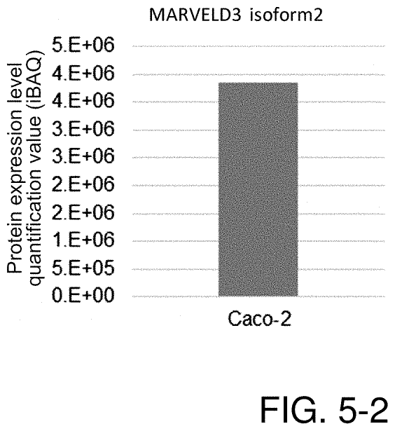

The expression of the selected candidate proteins is shown in FIGS. 1-1 to 1-16. In analyzing the colorectal cancer samples, CEACAM5 (SEQ ID NO: 10), which is an antigen currently undergoing clinical trials, serves as an index for comparing antigen expression profiles. CEACAM5 showed certain levels of expression in the normal tissues and the adjacent normal tissues, but even higher expression in almost all cases of cancer. NOX1, MARVELD3, and MANSC1 showed a different type of expression profile from that of CEACAM5, as their protein expression was not observed in normal tissues but was high (above the median value) in three to four cases of colorectal cancer tissue with a KRAS mutation, which showed large differences from the expression levels in the normal tissues adjacent to colorectal cancer tissue. SPINT2, SLC12A2, and CDCP1 showed a similar expression profile to that of CEACAM5, as they showed certain levels of expression in the normal tissues and the normal tissues adjacent to colorectal cancer tissue but even higher expression in the colorectal cancer tissues. In analyzing the lung cancer samples, PVRL4 (SEQ ID NO: 11), which is under ongoing clinical trials, serves as an index for comparing antigen expression profiles. PVRL4 and XPR1 were very highly cancer-specific antigens, with no protein expression observed in the normal tissues but high expression in the lung cancer tissues. TMPRSS4 and TNFRSF21 were not expressed in the normal tissues, but showed moderate expression levels in the lung cancer tissues. SLC7A5, STEAP1, FLVCR1, SEZ6L2, MMP14 and SPINT2 showed certain levels of expression in the normal tissues and the normal tissues adjacent to lung cancer tissue, but even higher expression in the lung cancer tissues.

### (Example 3) Isoform Expression Analysis of Target Antigen Candidate MARVELD3 from Colorectal Cancer

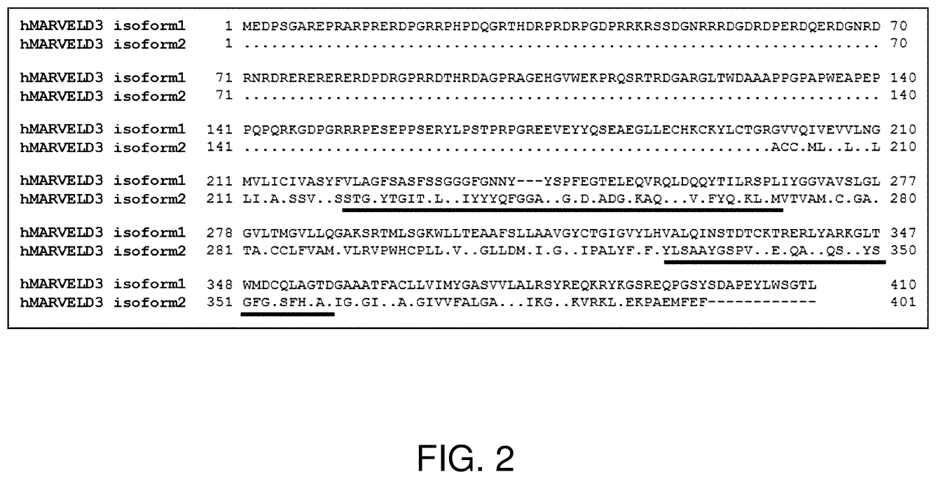

MARVELD3 is known to have isoforms 1 and 2, which have very similar intracellular region amino acid sequences but largely different extracellular regions (FIG. 2).

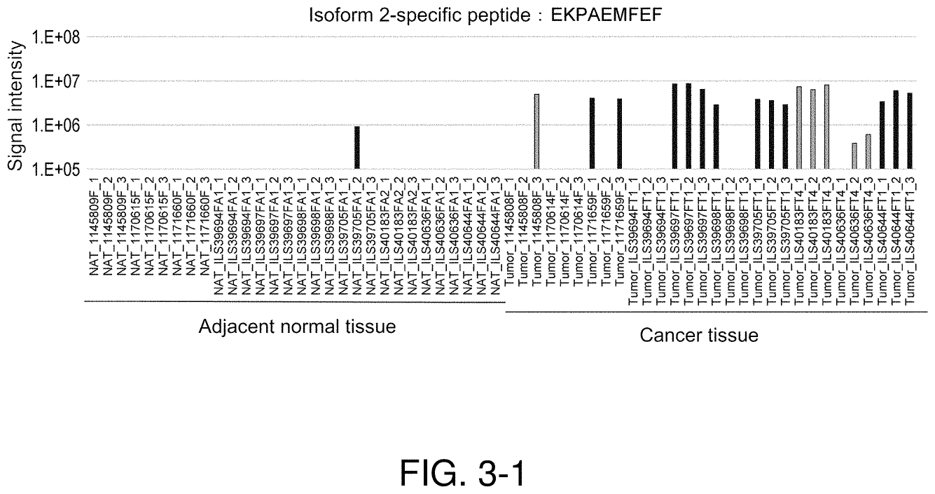

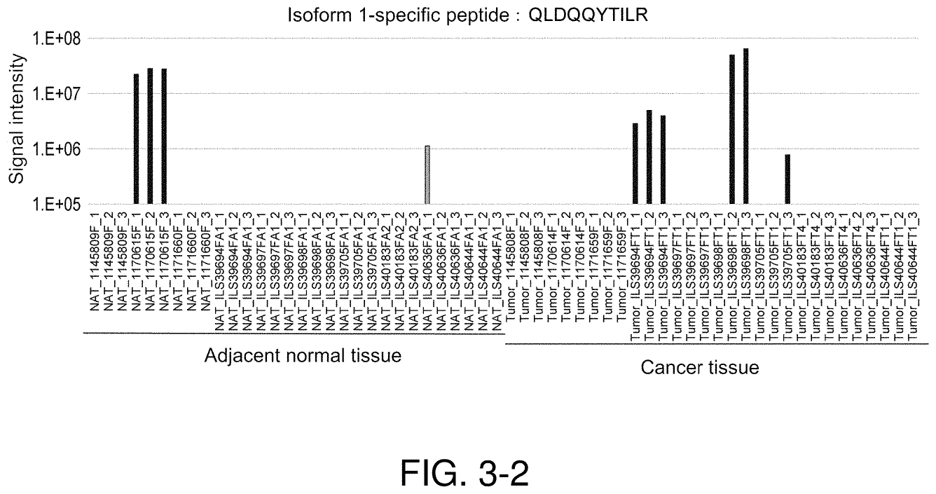

To determine which isoform is highly expressed in cancer tissue, the LC-MS peak intensities of the MARVELD3 peptide fragments identified in the proteomics analysis were examined. For the list of identified peptides and quantification values, one of the MaxQuant output files analyzed in Example 1-2, ModificationSpecificPeptides.txt, was used. In addition to the peptide fragments common to isoforms 1 and 2, the isoform 2-specific fragment was detected in four out of the seven colorectal cancer tissue specimens with a KRAS mutation (in two or more of three LC-MS runs), but in none of the adjacent normal tissues in two or more of three runs (FIG. 3-1). The isoform 1-specific fragment was detected in two out of the seven colorectal cancer tissue specimens with a KRAS mutation (FIG. 3-2). Therefore, it was presumed that isoform 2 is highly expressed in more colorectal cancer tissues with a KRAS mutation.

### (Example 4) Cell Surface Localization Analysis of Target Antigen Candidates

To examine the cell surface localization of XPR-1, MARVELD3 (isoform2), and NOX1, cell surface proteomics analysis was performed using Expi293 cells (ThermoFisher Scientific) made to overexpress myc-tag-added XPR-1 and MARVELD3 (isoform2) using ExpiFectamine (ThermoFisher Scientific) by the method recommended by the manufacturer, and C2BBe1, SW403, SW1463, DMS79 (ATCC), and HLC-1 cells (Riken), which presumably highly express these three proteins. After washing the cells with PBS, the cells were incubated with a cell membrane-impermeable biotin reagent (Sulfo-NHS-LC-biotin, ThermoFisher Scientific) to biotinylate the amino groups of the N-terminus and lysine side chains of cell surface proteins. The collected cells were ultrasonically disrupted using Covaris acoustic solubilizer or Bioruptor (Sonicbio) to obtain a cell extract.

Proteins in the cell extract were precipitated by the methanol/chloroform method and dissolved in an 8 M urea/400 mM ammonium bicarbonate solution. After reduction with dithiothreitol and alkylation with iodoacetamide, the proteins were digested with trypsin to obtain a peptide solution. The peptide solution was mixed with Neutravidin FG beads (Tamagawa Seiki), washed, and then eluted to purify biotinylated peptides.

The purified biotinylated peptides were analyzed using an LC-MS analysis system in which Q-Exactive (ThermoFisher Scientific) or Orbitrap fusion Lumos (ThermoFisher Scientific) was coupled to a nano-LC system (Ultimate 3000, Dionex). Raw data after the LC-MS analyses were used for database search using MaxQuant (http://www.biochem.mpg.de/5111795/maxquant) to identify biotinylated peptides and proteins and obtain quantification values. The database search was performed with the following parameters: Taxonomy: uniprot human, Fixed modification: carbamidomethylation (C), Variable modification: oxidation (M), Acetyl (Protein N-term), NHS-LC-biotin (N-term); NHS-LC-biotin (K), FDR (protein, peptide)<1%. For the list of identified peptides and quantification values, one of the MaxQuant output files, ModificationSpecificPeptides.txt, was used. The sequences and expression levels of the biotinylated peptides detected by this method enable determination of whether or not the protein to be analyzed is expressed on the cell membrane, and also enable identification of its extracellular region.

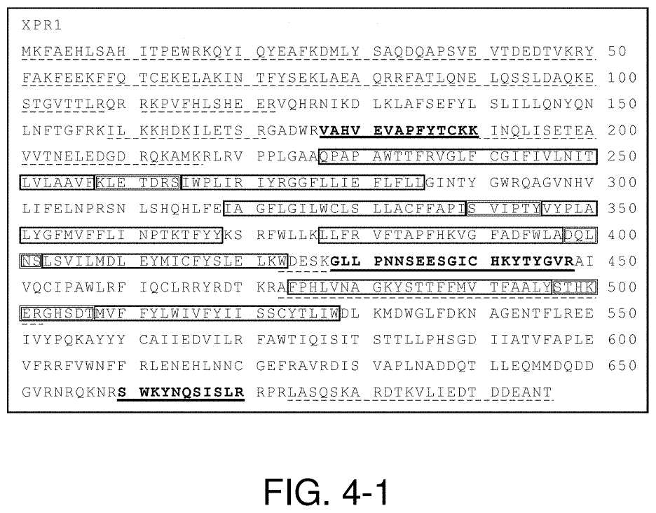

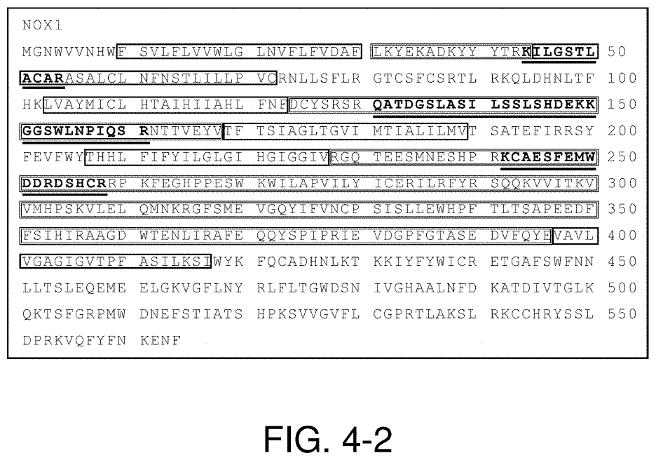

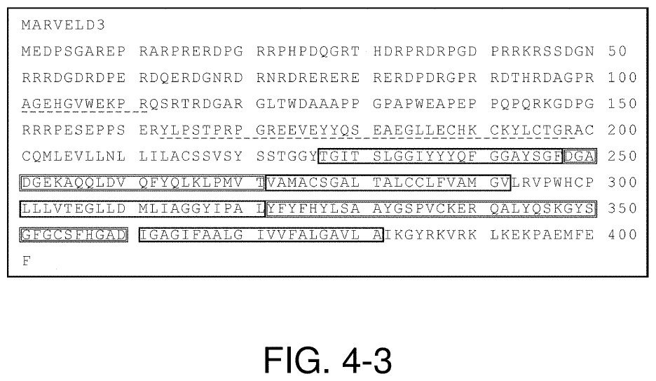

From cells overexpressing XPR1, biotinylated peptides of amino acid numbers 1-108, 111-122, 159-171, 177-216, 423-448, 473-502, 660-670, and 674-696 were detected (FIG. 4). In addition, biotinylated peptides of amino acid numbers 177-190, 428-448, and 660-670 were detected from the endogenous protein of HLC-1 or DMS-79 cells (FIG. 4-1). This confirmed that XPR1 is localized on the cell surface, and suggested that regions considered to be intracellular regions according to uniprot may be extracellular regions.

NOX1 was confirmed to be localized on the cell surface because biotinylated peptides of amino acid numbers 44-54, 131-161, and 242-258 were detected from C2BBe1, SW1463, or SW403 cells (FIG. 4-2).

For MARVELD3 isoform 2, biotinylated peptides of amino acid numbers 101-111 and 163-198 were detected from the overexpressing cells. However, no biotinylated peptide of the endogenous protein was detected from the analyzed cells (FIG. 4-3). The small number of lysin to be biotinylated in the putative extracellular region of MARVELD3 isoform 2 might make the detection of biotinylated peptides difficult.

### (Example 5) Protein Expression Analysis of Cell Lines (Proteomics Analysis)

HLC-1 cells (Riken), NCI-H2227 cells (ATCC), and Caco-2 cells (Riken) were suspended in a cell lysis buffer (1% NP-40, 50 mM Tris-Cl (pH7.5), 150 mM NaCl, Protease inhibitor cocktail (Roche)), and ultrasonically disrupted with an acoustic solubilizer (Covaris) to obtain cell extracts.

Proteins in the cell extracts were reduced with dithiothreitol, alkylated with iodoacetamide, and then precipitated by methanol/chloroform precipitation. The precipitated proteins were dissolved in 8 M urea/400 mM ammonium bicarbonate solution, diluted 4 times with distilled water, and then digested with lysyl endopeptidase and trypsin to obtain peptide solutions. The peptide solutions were desalted using Monospin C18 (GL Science) by the method recommended by the manufacturer. Thereafter, the desalted peptide solutions were fractionated using AssayMAP Bravo cartridge RP-S(Agilent) by the method recommended by the manufacturer, and each fraction was analyzed by an LC-MS analysis system in which Q-Exactive (ThermoFisher Scientific) was coupled to a nano-LC system (ThermoFisher Scientific).

Raw data after the LC-MS analyses were used for database search using MaxQuant (http://www.biochem.mpg.de/5111795/maxquant) to identify proteins and obtain quantification values (iBAQ, Intensity Based Absolute Quantification). The database search was performed with the following parameters:

Taxonomy: uniprot human, Fixed modification: carbamidomethylation (C), Variable modification: oxidation (M); deamidation (NQ), Acetyl (protein N-term), and FDR (protein, peptide)<1%.
The iBAQ values for XPR1 and MARVELD3 set forth in the MaxQuant output file “proteinGroups.txt” are shown in FIG. 5-1 and FIG. 5-2.

### (Example 6) Preparation of Anti-XPR1 Antibody

A plasmid vector expressing full-length human XPR1 (SEQ ID NO: 1) with a TT peptide sequence (FNNFTVSFWLRVPKVSASHLE, SEQ ID NO: 22) added to the C-terminus (human XPR1-TT, nucleotide SEQ ID NO: 23, amino acid SEQ ID NO: 24) was prepared. Four New Zealand White rabbits (Kitayama Labes) were immunized with the prepared plasmid vector six times by a gene gun method and an in vivo electroporation method. Peripheral blood mononuclear cells and spleen cells were harvested from the rabbits one week after the final immunization. Surface IgG-positive B cells were concentrated from the harvested rabbit-derived cells by MACS using PE-labeled anti-rabbit IgG antibody (Southern biotech) and cultured. B cells secreting an antibody binding to human XPR1 were identified from the cultured B cells using a fluorescence microscope attached to the micromanipulator CellCelector (ALS) and collected in a microwell plate using the micromanipulator CellCelector (ALS). Genes constituting the variable regions of rabbit immunoglobulin were cloned from the collected B cells by RT-PCR. These rabbit immunoglobulin variable region DNA fragments were inserted into an expression vector containing the IgG constant regions to prepare a recombinant antibody. The anti-human XPR1 antibody was prepared by a method known to those skilled in the art. The prepared anti-human XPR1 antibody is shown in Table 3.

TABLE 3

| Name of antibody | XPB0062 |
|---|---|
| HVR_H1 | SEQ ID NO: 35 |
| HVR_H2 | SEQ ID NO: 36 |
| HVR_H3 | SEQ ID NO: 37 |
| HVR_L1 | SEQ ID NO: 38 |
| HVR_L2 | SEQ ID NO: 39 |
| HVR_L3 | SEQ ID NO: 40 |
| VH | SEQ ID NO: 41 |
| VL | SEQ ID NO: 42 |

As an anti-human CD3 antibody, CE115TR (heavy chain variable region SEQ ID NO: 65, light chain variable region SEQ ID NO: 66), which binds to the CD3c chain constituting the T cell receptor and induces an activation signal of the T cell receptor, was prepared by a method known to those skilled in the art. The present inventors prepared a bispecific antibody consisting of an anti-human XPR1 antibody and an anti-CD3 antibody by a method known to those skilled in the art. The prepared anti-human XPR1/anti-human CD3 bispecific antibody is shown in Table 4. The heavy chain constant region sequence on the anti-human XPR1 side of the bispecific antibody is the sequence shown in SEQ ID NO: 83, the light chain constant region sequence on the anti-human XPR1 side is the sequence shown in SEQ ID NO: 85, the heavy chain constant region sequence on the anti-human CD3 side is the sequence shown in SEQ ID NO: 84, and the light chain constant region sequence on the anti-human CD3 side is the sequence shown in SEQ ID NO: 86.

TABLE 4

| Name of Antibody | | TR01H113//XPB0062HCa |
|---|---|---|
| Anti-human | HVR_H1 | SEQ ID NO: 35 |
| XPR1 | HVR_H2 | SEQ ID NO: 36 |
| | HVR_H3 | SEQ ID NO: 37 |
| | HVR_L1 | SEQ ID NO: 38 |
| | HVR_L2 | SEQ ID NO: 39 |
| | HVR_L3 | SEQ ID NO: 40 |
| | VH | SEQ ID NO: 41 |
| | VL | SEQ ID NO: 42 |
| Anti-human | HVR_H1 | SEQ ID NO: 59 |
| CD3 | HVR_H2 | SEQ ID NO: 60 |
| | HVR_H3 | SEQ ID NO: 61 |
| | HVR_L1 | SEQ ID NO: 62 |
| | HVR_L2 | SEQ ID NO: 63 |
| | HVR_L3 | SEQ ID NO: 64 |
| | VH | SEQ ID NO: 65 |
| | VL | SEQ ID NO: 66 |

### (Example 7) Confirmation of Binding of Anti-Human XPR1 Antibody to Human XPR1

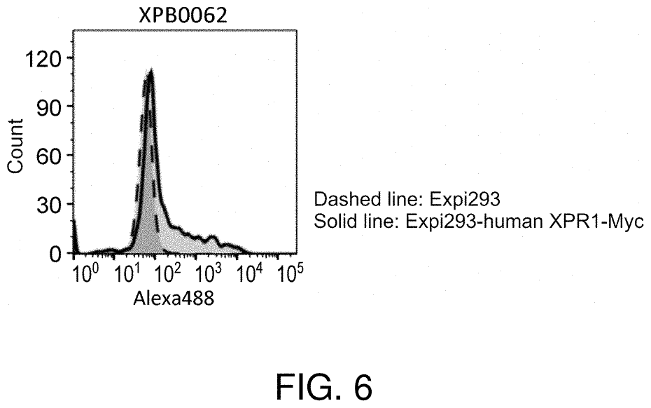

A plasmid vector expressing in mammalian cells full-length human XPR1 (amino acid SEQ ID NO: 1) with a Myc tag sequence (EQKLISEEDL, SEQ ID NO: 25) added to the C-terminus (human XPR1-Myc, nucleotide SEQ ID NO: 26, amino acid SEQ ID NO: 27) was constructed. The protein was transiently expressed in Expi293 cells (Thermo Fisher Scientific) by introducing the constructed plasmid vector into Expi293 cells. At the same time, Expi293 cells transfected with an empty plasmid vector were also prepared as a control. The Expi293 cells transfected with an empty plasmid vector were stained with CellTrace FarRed (Thermo Fisher Scientific) and then mixed with the Expi293 cells made to express human XPR1-Myc, and subsequently reacted with the anti-human XPR1 antibody prepared in Example 6. Then, anti-human IgG Fc cross-adsorbed Alexa488 (Invitrogen) was reacted to stain the anti-human XPR1 antibody. Dead cells were stained with DAPI (sigma-aldrich). The cells reacted with the antibody were measured by FACS Aria III (Becton, Dickinson and Company). The obtained data were analyzed by FlowJo ver.10. The live cell fraction, which was not stained with DAPI fluorescence, was separated into a fraction that was stained with CellTrace FarRed (cells transfected with the empty plasmid vector) and a fraction that was not stained with CellTrace FarRed (cells transfected with the human XPR1-Myc expression plasmid vector), and Alexa488 staining for each fraction was examined. As a result, more intense Alexa488 staining was observed in the fraction that was not stained with CellTrace FarRed (cells transfected with the human XPR1-Myc expression plasmid vector) than in the fraction that was stained with CellTrace FarRed (cells transfected with an empty plasmid vector) (FIG. 6). This result confirmed that the anti-human XPR1 antibody prepared in Example 6 binds to human XPR1 expressed on cells.

### (Example 8) Cytotoxic Activity Assay of Anti-Human XPR1/Anti-Human CD3 Bispecific Antibody

### LDH Cytotoxicity Assay Using Human Peripheral Blood Mononuclear Cells (PBMC)

The cytotoxic activity of the anti-human XPR1/anti-human CD3 bispecific antibody prepared in Example 6 was evaluated by the LDH release method (LDH Cytotoxicity Detection Kit: TAKARA). First, the antibody solutions at various concentrations (0.04, 0.004, and 0.0004 mg/mL) diluted with the culture solution (RPMI1640 solution supplemented with 10% FBS) to make 4 times of the final concentration were added to a 96-well U-bottom plate at 50 μL per well. Next, as target cells, HLC-1 was adjusted to 1×10⁵ cells/mL, NCI-H2227 was adjusted to 2×10⁵ cells/mL. The cells were seeded at 100 μL/well or 50 μL/well, respectively, so that the final number of cells would be 1×10⁴ cells/well, and the U-bottom plate was allowed to stand at room temperature for 15 minutes. As effector cells, human PBMCs (peripheral blood mononuclear cells) were adjusted to 4×10⁶ cells/mL or 2×10⁶ cells/mL. The cells of 4×10⁶ cells/mL were added to the wells seeded with HLC-1 cells, and the cells of 2×10⁶ cells/mL to the wells seeded with NCI-H2227 cells, at 50 μL per well. Subsequently, the U-bottom plate was allowed to stand at 37° C. for approximately 24 hours in a 5% carbon dioxide gas incubator, and then centrifuged. In the U-bottom plate, in addition to the wells to which the above target cells, the effector cells, and the antibody were added, wells to which only the culture solution was added, wells to which only the target cells and the effector cells were added (no antibody added), and wells to which the target cells, the effector cells, and Triton X-100 were added (no antibody added), were also prepared.

One hundred μL of culture supernatant in each well of the U bottom plate was transferred to a 96-well flat bottom plate. The catalyst solution was dissolved in 1 mL of H₂O and mixed with the dye solution to a catalyst solution to dye solution ratio of 1:45. The mixture of the catalyst solution and the dye solution was dispensed into the 96-well flat bottom plate to which the culture supernatant had been transferred, and the flat bottom plate was allowed to stand at room temperature for 15 to 30 minutes.

The absorbance at 492 nm was measured for each well, and the absorbance at the control wavelength of 620 nm measured at the same time was used to calculate 492-620 nm absorbance. The 492-620 nm absorbance of each well was applied to the following formula to calculate the cell growth inhibition rate (Cytotoxicity).

$$Cell growth inhibition rate ( \% ) = \frac{( A - D ) - ( B - D )}{( C - D ) - ( B - D )} \times 100$$

A: 492-620 nm absorbance of wells to which target cells, effector cells, and antibody were added
B: 492-620 nm absorbance of wells to which only target cells and effector cells were added
C: 492-620 nm absorbance of wells to which target cells, effector cells, and Triton X-100 were added
D: mean 492-620 nm absorbance of wells containing only the culture medium (blank)

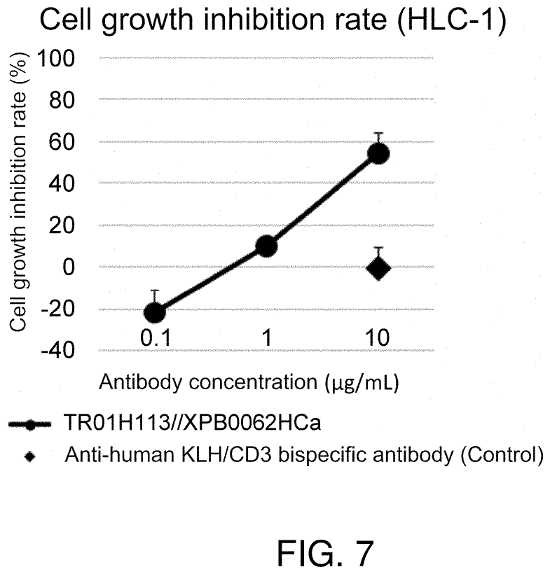

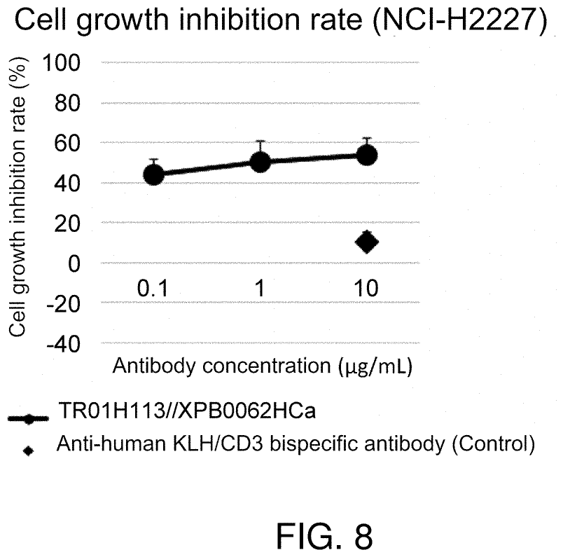

The anti-human XPR1/anti-human CD3 bispecific antibody showed a dose-dependent cell growth inhibitory activity (FIGS. 7 and 8).

### (Example 9) Preparation of Anti-Human MARVELD3 Isoform2 Antibody

A plasmid vector expressing human MARVELD3 isoform2 (nucleotide SEQ ID NO: 28, amino acid SEQ ID NO: 4) was constructed. Four New Zealand White rabbits (Kitayama Labes) were immunized with the constructed plasmid vector six times by a gene gun method and an in vivo electroporation method, and peripheral blood mononuclear cells and spleen cells were harvested from the rabbits one week after the final immunization. Surface IgG-positive B cells were concentrated from the harvested rabbit-derived cells by MACS using PE-labeled anti-rabbit IgG antibody (Southern biotech), and cultured. B cells secreting antibodies binding to human MARVELD3 isoform2 were identified from the cultured B cells using a fluorescence microscope attached to the micromanipulator CellCelector (ALS) and collected in a microwell plate using the micromanipulator CellCelector (ALS). Genes constituting the variable regions of rabbit immunoglobulin were cloned from the collected B cells by RT-PCR. These rabbit immunoglobulin variable region DNA fragments were inserted into an expression vector containing the IgG constant regions to prepare recombinant antibodies. The anti-human MARVELD3 isoform2 antibodies were prepared by a method known to those skilled in the art. The prepared anti-human MARVELD3 isoform2 antibodies are shown in Table 5.

TABLE 5

| Name of antibody | MDA0279 | MDA0314 |
|---|---|---|
| HVR_H1 | SEQ ID NO: 43 | SEQ ID NO: 51 |
| HVR_H2 | SEQ ID NO: 44 | SEQ ID NO: 52 |
| HVR_H3 | SEQ ID NO: 45 | SEQ ID NO: 53 |
| HVR_L1 | SEQ ID NO: 46 | SEQ ID NO: 54 |
| HVR_L2 | SEQ ID NO: 47 | SEQ ID NO: 55 |
| HVR_L3 | SEQ ID NO: 48 | SEQ ID NO: 56 |
| VH | SEQ ID NO: 49 | SEQ ID NO: 57 |
| VL | SEQ ID NO: 50 | SEQ ID NO: 58 |

As an anti-human CD3 antibody, CE115TR (heavy chain variable region SEQ ID NO: 65, light chain variable region SEQ ID NO: 66), which binds to the CD3ε chain constituting the T cell receptor and induces an activation signal of the T cell receptor, was prepared by a method known to those skilled in the art. The present inventors prepared bispecific antibodies consisting of an anti-human MARVELD3 isoform2 antibody and an anti-CD3 antibody by a method known to those skilled in the art. The prepared anti-human MARVELD3 isoform2/anti-human CD3 bispecific antibodies are shown in Table 6. The heavy chain constant region sequence on the anti-human MARVELD3 isoform2 side of the bispecific antibodies is the sequence shown in SEQ ID NO: 83, the light chain constant region sequence on the anti-human MARVELD3 isoform2 side is the sequence shown in SEQ ID NO: 85, the heavy chain constant region sequence on the anti-human CD3 side is the sequence shown in SEQ ID NO: 84, and the light chain constant region sequence of the anti-human CD3 side is the sequence shown in SEQ ID NO: 86.

TABLE 6

| | | TR01H113// | TR01H113// |
|---|---|---|---|
| Name of Antibody | | MDA0279 | MDA0314 |
| Anti-human | HVR_H1 | SEQ ID NO: 43 | SEQ ID NO: 51 |
| MARVELD3 | HVR_H2 | SEQ ID NO: 44 | SEQ ID NO: 52 |
| isoform2 | HVR_H3 | SEQ ID NO: 45 | SEQ ID NO: 53 |
| | HVR_L1 | SEQ ID NO: 46 | SEQ ID NO: 54 |
| | HVR_L2 | SEQ ID NO: 47 | SEQ ID NO: 55 |
| | HVR_L3 | SEQ ID NO: 48 | SEQ ID NO: 56 |
| | VH | SEQ ID NO: 49 | SEQ ID NO: 57 |
| | VL | SEQ ID NO: 50 | SEQ ID NO: 58 |

| | | |
|---|---|---|
| Anti-human | HVR_H1 | SEQ ID NO: 59 |
| CD3 | HVR_H2 | SEQ ID NO: 60 |
| | HVR_H3 | SEQ ID NO: 61 |
| | HVR_L1 | SEQ ID NO: 62 |
| | HVR_L2 | SEQ ID NO: 63 |
| | HVR_L3 | SEQ ID NO: 64 |
| | VH | SEQ ID NO: 65 |
| | VL | SEQ ID NO: 66 |

### (Example 10) Confirmation of Binding of Anti-Human MARVELD3 Isoform2 Antibodies to MARVELD3 Isoform2

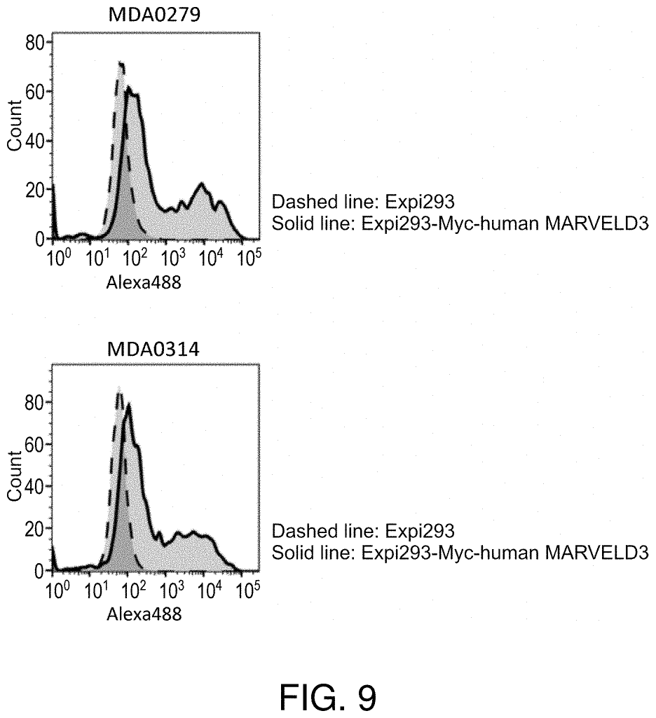

A plasmid vector that expresses, in mammalian cells, a molecule (Myc-human MARVELD3, nucleotide SEQ ID NO: 29, amino acid SEQ ID NO: 30) in which the Myc tag sequence (EQKLISEEDL, SEQ ID NO: 25) has been added after the N-terminal methionine of full-length human MARVELD3 isoform2 (amino acid SEQ ID NO: 4), was introduced into Expi293 cells (Thermo Fisher Scientific), and the protein was transiently expressed in the Expi293 cells. At the same time, Expi293 cells transfected with an empty plasmid vector were also prepared as a control. The Expi293 cells transfected with an empty plasmid vector were stained with CellTrace FarRed (Thermo Fisher Scientific) and then mixed with the Expi293 cells made to express human MARVELD3 isoform2, and subsequently reacted with an anti-human MARVELD3 isoform2 antibody prepared in Example 9. Then, anti-human IgG Fc cross-adsorbed Alexa488 (Invitrogen) was reacted to stain the anti-human MARVELD3 isoform2 antibody. Dead cells were stained with DAPI (sigma-aldrich). The cells reacted with the antibody were measured by FACS Aria III (Becton, Dickinson and Company). The obtained data were analyzed by FlowJo ver.10. The live cell fraction, which was not stained with DAPI fluorescence, was separated into a fraction that was stained with CellTrace FarRed (cells transfected with the empty plasmid vector) and a fraction that was not stained with CellTrace FarRed (cells transfected with the human XPR1-Myc expression plasmid vector), and Alexa488 staining for each fraction was examined. As a result, more intense Alexa488 staining was observed in the fraction that was not stained with CellTrace FarRed (cells transfected with the human MARVELD3 isoform2 expression plasmid vector) than in the fraction that was stained with CellTrace FarRed (cells transfected with the empty plasmid vector) (FIG. 9). This result confirmed that the anti-human MARVELD3 isoform2 antibodies prepared in Example 9 bind to human MARVELD3 isoform2 expressed on cells.

### (Example 11) Cytotoxic Activity Assay of Anti-Human MARVELD3 Isoform2/Anti-Human CD3 Bispecific Antibody

Cytotoxic activity assay using human peripheral blood mononuclear cells

The cytotoxic activity of the anti-human MARVELD3 isoform2/anti-human CD3 bispecific antibodies prepared in Example 9 was evaluated based on cell growth inhibition rates determined using xCELLigence Real-Time Cell Analyzer (Roche Diagnostics). The target cells used were Caco-2 cells. First, 50 μL of the medium was added to each well of the E-Plate 96 (Roche Diagnostics) plate. Caco-2 cells were detached from the dish and seeded at 1.3×10³ cells/well (50 μl/well). The live cell assay was then initiated using the xCELLigence Real-Time Cell Analyzer. On the following day, the plate was removed from the xCELLigence Real-Time Cell Analyzer and 50 μL of each antibody prepared at various concentrations (0.1, 1.0, and 10 μg/mL) was added to each well of the plate. In addition, 50 μL of human PBMC suspension (5×10⁴ cells/well) was added. The plate was placed again on the xCELLigence Real-Time Cell Analyzer and live cell assay was initiated. The reaction was carried out at 37° C. under 5% carbon dioxide gas. The cell growth inhibition rate (%) was determined according to the following equation using cell index values 72 hours after addition of human PBMC. The cell index values used for the calculation were normalized such that the cell index value immediately before addition of the antibody was 1.

$$Cell growth inhibition rate ( \% ) = \frac{A - B}{A - 1} \times 100$$

A represents the mean cell index value of antibody-free wells (only target cells and human PBMC), and B represents the mean cell index value of each well. The measurement was repeated three times.

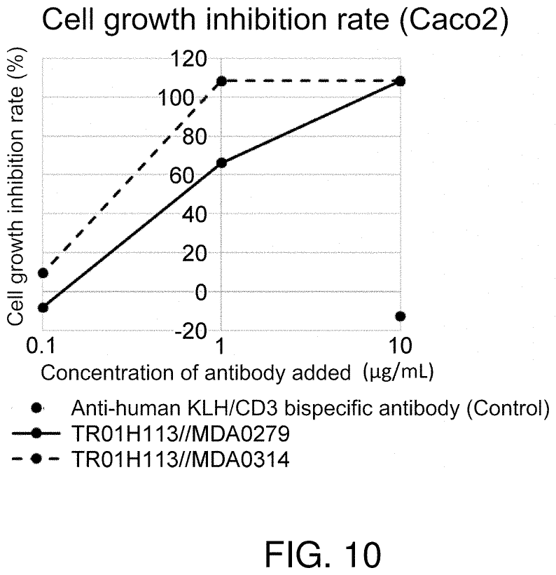

The cytotoxic activity of the anti-KLH/CD3 bispecific antibodies, TR01H113//MDA0279 and TR01H113//MDA0314, was measured using peripheral blood mononuclear cells (PBMC). TR01H113//MDA0279 and TR01H113//MDA0314 showed high cell growth inhibition rates in comparison to the control anti-KLH/CD3 bispecific antibody (FIG. 10).

### (Example 12) Preparation of Human NOX1 Protein

A gene encoding the full-length human NOX1 sequence (amino acid SEQ ID NO: 2) with a Twin-Strep tag sequence (WSHPQFEKGGGSGGGSGGSAWSHPQFEK, SEQ ID NO: 31) added to the C-terminus (human NOX1-Strep, amino acid SEQ ID NO: 34) was synthesized, and cloned into a mammalian cell expression vector. The protein was transiently expressed by introducing this expression vector into Expi293 cells (Thermo Fisher Scientific).

The obtained cells were collected by centrifugation, and Fos-choline 14 and cholesterol hemisuccinate were added at final concentrations of 1% and 0.2%, respectively, to solubilize the human NOX1-Strep protein. The solubilized protein sample was purified by affinity chromatography using Strep-Tactin XT Superflow (IBA GmbH). Fractions containing the human NOX1-Strep protein were collected and Amphipol A8-35 solution (Anatrace) was added at a final concentration of 2 mg/mL. Immediately after that, Bio-Beads SM-2 Adsorbent Media (BIO-RAD) was added and incubated at 4° C. for 12-16 hours to remove Fos-choline 14/cholesterol hemisuccinate and reconstitute the human NOX1-Strep protein into Amphipol A8-35. The reconstituted human NOX1-Strep protein sample was purified by gel filtration chromatography using a Superdex 200 increase 10/300 column (GE Healthcare). Fractions containing the human NOX1-Strep protein were collected and concentrated using a 50 kDa MWCO ultrafiltration membrane. To the concentrated sample, Amphipol A8-35 was added at a final concentration of 2 mg/mL to obtain a final preparation.

### (Example 13) Preparation of Anti-Human NOX1 Antibodies

Two New Zealand White rabbits (Kitayama Labes) were immunized four times using the human NOX1-Strep protein prepared in Example 12, and peripheral blood mononuclear cells and spleen cells were harvested from the rabbits 6 days after the final immunization. A gene encoding amino acids 1 to 564 of human NOX1 (SEQ ID NO: 2) with the signal sequence and the transmembrane region of Neurexin 1B-delta fused to the N-terminus (Cell. 2018 Nov. 1, 175(4):1131-1140.e11.) and a Myc tag sequence (EQKLISEEDL) added to the C-terminus (NOX1_Nx1B_564-myc, nucleotide SEQ ID NO: 32, amino acid SEQ ID NO: 33) was synthesized, and this was cloned into a mammalian cell plasmid expression vector. Using the constructed plasmid vector, eight New Zealand White rabbits (Kitayama Labes) were immunized six times by a gene gun method and a Tropis (PharmaJet) device, and spleen cells were harvested from the rabbits six or seven days after the final immunization. Surface IgG-positive B cells were concentrated from the harvested rabbit-derived cells by MACS and FACS using PE-labeled anti-rabbit IgG antibody (Southern biotech), and cultured. B cells secreting an antibody that binds to human NOX1 were identified from the cultured B cells using a fluorescence microscope attached to the micromanipulator CellCelector (ALS) and collected in a microwell plate using the micromanipulator CellCelector (ALS). Genes constituting the variable regions of rabbit immunoglobulin were cloned from the collected B cells by RT-PCR. These rabbit immunoglobulin variable region DNA fragments were inserted into an expression vector containing IgG constant regions to prepare recombinant antibodies. The anti-human NOX1 antibodies were prepared by a method known to those skilled in the art. The prepared anti-human NOX1 antibodies are shown in Table 7.

TABLE 7

| Name of antibody | NXA0125 | NXA0164 |
|---|---|---|
| HVR_H1 | SEQ ID NO: 67 | SEQ ID NO: 75 |
| HVR_H2 | SEQ ID NO: 68 | SEQ ID NO: 76 |
| HVR_H3 | SEQ ID NO: 69 | SEQ ID NO: 77 |
| HVR_L1 | SEQ ID NO: 70 | SEQ ID NO: 78 |
| HVR_L2 | SEQ ID NO: 71 | SEQ ID NO: 79 |
| HVR_L3 | SEQ ID NO: 72 | SEQ ID NO: 80 |
| VH | SEQ ID NO: 73 | SEQ ID NO: 81 |
| VL | SEQ ID NO: 74 | SEQ ID NO: 82 |

As an anti-human CD3 antibody, CE115TR (heavy chain variable region SEQ ID NO: 65, light chain variable region SEQ ID NO: 66), which binds to the CD3c chain constituting the T cell receptor and induces an activation signal of the T cell receptor, was prepared by a method known to those skilled in the art. The present inventors prepared bispecific antibodies consisting of an anti-human NOX1 antibody and an anti-CD3 antibody by a method known to those skilled in the art. The prepared anti-human NOX1/anti-human CD3 bispecific antibodies are shown in Table 8. The heavy chain constant region sequence on the anti-human NOX1 side of the bispecific antibodies is the sequence shown in SEQ ID NO: 83, the light chain constant region sequence on the anti-human XPR1 side is the sequence shown in SEQ ID NO: 85, the heavy chain constant region sequence on the anti-human CD3 side is the sequence shown in SEQ ID NO: 84, and the light chain constant region sequence on the anti-human CD3 side is the sequence shown in SEQ ID NO: 86.

TABLE 8

| Name of Antibody | | TR01H113//NXA0125 | TR01H113//NXA0164 |
|---|---|---|---|
| Anti-human | HVR_H1 | SEQ ID NO: 67 | SEQ ID NO: 75 |
| NOX1 | HVR_H2 | SEQ ID NO: 68 | SEQ ID NO: 76 |
| | HVR_H3 | SEQ ID NO: 69 | SEQ ID NO: 77 |
| | HVR_L1 | SEQ ID NO: 70 | SEQ ID NO: 78 |
| | HVR_L2 | SEQ ID NO: 71 | SEQ ID NO: 79 |
| | HVR_L3 | SEQ ID NO: 72 | SEQ ID NO: 80 |
| | VH | SEQ ID NO: 73 | SEQ ID NO: 81 |
| | VL | SEQ ID NO: 74 | SEQ ID NO: 82 |

| | | |
|---|---|---|
| Anti-human | HVR_H1 | SEQ ID NO: 59 |
| CD3 | HVR_H2 | SEQ ID NO: 60 |
| | HVR_H3 | SEQ ID NO: 61 |
| | HVR_L1 | SEQ ID NO: 62 |
| | HVR_L2 | SEQ ID NO: 63 |
| | HVR_L3 | SEQ ID NO: 64 |
| | VH | SEQ ID NO: 65 |
| | VL | SEQ ID NO: 66 |

### (Example 14) Confirmation of Binding of Anti-Human NOX1 Antibodies to Human NOX1

A plasmid vector expressing human NOX1-Strep (amino acid SEQ ID NO: 34) or a plasmid vector expressing NOX1_Nx1B_564-myc (amino acid SEQ ID NO: 33) was introduced into Expi293 cells (Thermo Fisher Scientific) to express the protein transiently. At the same time, Expi293 cells transfected with an empty plasmid vector were also prepared as a control. These cells were treated with 4% paraformaldehyde and 1% digitonin. The Expi293 cells transfected with an empty plasmid vector were stained with CellTrace FarRed (Thermo Fisher Scientific), the Expi293 cells transfected with the plasmid vector expressing human NOX1-Strep were stained with CellTrace FarRed and CellTrace Violet (Thermo Fisher Scientific), and then mixed with the Expi293 cells made to express NOX1_Nx1B_564-myc. Subsequently, the cells were reacted with an anti-human NOX1 antibody prepared in Example 13.

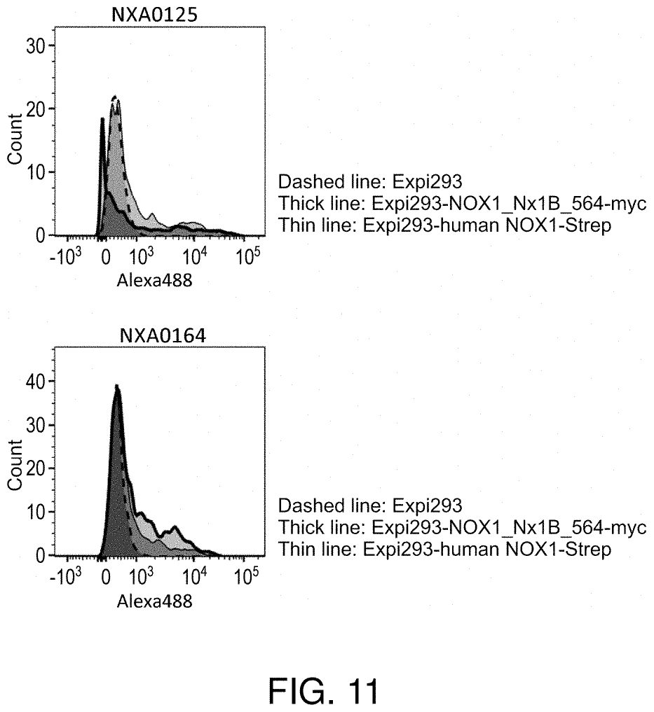

Then, anti-human IgG Fc cross-adsorbed Alexa488 (Invitrogen) was reacted to stain the anti-human NOX1 antibody. The cells reacted with the antibody were measured by FACS Aria III (Becton, Dickinson and Company). The obtained data were analyzed by FlowJo ver.10. Fractions stained with CellTrace FarRed (cells transfected with the empty plasmid vector), not stained (cells transfected with human NOX1-Strep), and stained with CellTrace FarRed and CellTrace Violet (cells transfected with NOX1_Nx1B_564-myc) were separated, and Alexa488 staining for each fraction was examined More intense staining was observed in the unstained fraction (cells transfected with human NOX1-Strep) and the fraction stained with CellTrace FarRed and CellTrace Violet (cells transfected with NOX1_Nx1B_564-myc) than in the fraction stained with CellTrace FarRed (cells transfected with the empty plasmid vector) (FIG. 11). This result confirmed that the anti-human NOX1 antibodies prepared in Example 13 bind to human NOX1 expressed on cells.

Although the foregoing invention has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

### INDUSTRIAL APPLICABILITY

The antibodies of the present disclosure have at least one of cytotoxic activity and internalization activity, and are useful for either or both treatment and prevention of cancer (for example, lung cancer and colorectal cancer).

1. An isolated antibody that binds to any of the following proteins:
XPR1, NOX1, MARVELD3 isoform 1, MARVELD3 isoform 2, SPINT2, MANSC1, SLC12A2, CDCP1, SEZ6L2, FLVCR1, SLC7A5, STEAP1, MMP14, TNFRSF21, and TMPRSS4.

2. The antibody of claim 1, which binds to an extracellular domain of the protein.

3. The antibody of claim 1 or 2, which binds to any of:
(1) XPR1 represented by SEQ ID NO: 1,
(2) NOX1 represented by SEQ ID NO: 2,
(3) MARVELD3 isoform 1 represented by SEQ ID NO: 3,
(4) MARVELD3 isoform 2 represented by SEQ ID NO: 4,
(5) SPINT2 represented by SEQ ID NO: 5,
(6) MANSC1 represented by SEQ ID NO: 7,
(7) SLC12A2 represented by SEQ ID NO: 8,
(8) CDCP1 represented by SEQ ID NO: 9,
(9) SEZ6L2 represented by SEQ ID NO: 12,
(10) FLVCR1 represented by SEQ ID NO: 13,
(11) SLC7A5 represented by SEQ ID NO: 14,
(12) STEAP1 represented by SEQ ID NO: 15,
(13) MMP14 represented by SEQ ID NO: 16,
(14) TNFRSF21 represented by SEQ ID NO: 17, and
(15) TMPRSS4 represented by SEQ ID NO: 18.

4. The antibody of claim 1 or 2, which binds to any of:
(1) an extracellular domain of XPR1, which is any of amino acids 1 to 108, 111 to 122, 159 to 171, 177 to 216, 423 to 448, 473 to 502, 660 to 670, 674 to 696, 177 to 190, 428 to 448, 660 to 670, 244, 256 to 273, 257 to 270, 258 to 264, 258 to 268, 256 to 270, 258 to 273, 260 to 270, 293 to 314, 336 to 343, 337 to 344, 338 to 341, 340 to 342, 340 to 344, 343 to 344, 340 to 345, 368 to 372, 392 to 398, 397 to 401, 398 to 402, 420 to 442, 420 to 506, 465 to 479, 497 to 507, 498 to 508, 529 to 555, 529 to 570, 582 to 586, 1 to 234, 1 to 236, 293 to 318, 367 to 442, 369 to 473, 500 to 507, 529 to 696, and 589 to 696 in the amino acid sequence represented by SEQ ID NO: 1;
(2) an extracellular domain of NOX1, which is any of amino acids 44 to 54, 131 to 161, 242 to 258, 1 to 4, 1 to 11, 18 to 55, 28 to 44, 31 to 44, 32 to 46, 34 to 50, 70 to 102, 70 to 103, 117 to 176, 120 to 172, 122 to 166, 122 to 172, 124 to 168, 190 to 208, 191 to 204, 223 to 266, 223 to 267, 227 to 269, 228 to 391, 228 to 396, 404, and 420 to 564 in the amino acid sequence represented by SEQ ID NO: 2;
(3) an extracellular domain of MARVELD3 isoform 1, which is any of amino acids 222 to 266, 223 to 269, 227 to 264, 227 to 268, 229 to 263, 231 to 265, 321 to 362, 322 to 357, 323 to 357, 324 to 357, and 324 to 358 in the amino acid sequence represented by SEQ ID NO: 3; and
(4) an extracellular domain of MARVELD3 isoform 2, which is any of amino acids 101 to 111, 163 to 198, 1 to 266, 1 to 270, 216 to 271, 222 to 269, 226 to 271, 227 to 268, 227 to 271, 248 to 271, 316 to 364, 322 to 360, 323 to 359, 324 to 360, 326 to 360, and 327 to 360 in the amino acid sequence represented by SEQ ID NO: 4.

5. The antibody of any one of claims 1 to 4, which has cytotoxic activity.

6. The antibody of any one of claims 1 to 5, which is a multispecific antibody.

7. The antibody of claim 6, further comprising a T cell receptor complex-binding domain.

8. The antibody of claim 6 or 7, comprising an Fc region with reduced Fcγ receptor-binding activity.

9. The antibody of claim 7 or 8, wherein the antibody has cytotoxic activity, and the cytotoxic activity is a T cell-dependent cytotoxic activity.

10. The antibody of any one of claims 7 to 9, wherein the T cell receptor complex-binding domain is a T cell receptor-binding domain having T cell receptor-binding activity.

11. The antibody of any one of claims 7 to 9, wherein the T cell receptor complex-binding domain is a CD3-binding domain having CD3-binding activity.

12. A pharmaceutical formulation comprising the antibody of any one of claims 1 to 11 and a pharmaceutically acceptable carrier.

13. The antibody of any one of claims 1 to 11 for use in treatment of cancer.

14. The antibody of claim 13, wherein the cancer is lung cancer, and the antibody binds to any of XPR1, SPINT2, SEZ6L2, FLVCR1, SLC7A5, STEAP1, MMP14, TNFRSF21, and TMPRSS4.

15. The antibody of claim 13, wherein the cancer is colorectal cancer, and the antibody binds to any of NOX1, MARVELD3 isoform 1, MARVELD3 isoform 2, SPINT2, MANSC1, SLC12A2, and CDCP1.
